(19) United States
(12) Patent Application Publication
(10) Pub. No.: US 2013/0197480 A1
(43) Pub. Date: Aug. 1, 2013

(54) METHODS AND SYSTEMS FOR DELIVERY OF LIVE CELLS

(71) Applicant: Palomar Medical Technologies, Inc., Burlington, MA (US)

(72) Inventor: David H. McDaniel, Virginia Beach, VA (US)

(73) Assignee: PALOMAR MEDICAL TECHNOLOGIES, INC., Burlington, MA (US)

(21) Appl. No.: 13/754,580

(22) Filed: Jan. 30, 2013

Related U.S. Application Data
(60) Provisional application No. 61/593,193, filed on Jan. 31, 2012.
(60) Provisional application No. 61/716,426, filed on Oct. 19, 2012.

(51) Int. Cl. A61M 37/00

(52) U.S. Cl. CPC A61M 37/00
USPC 604/522

(57) ABSTRACT

Described herein are methods for delivering live cells into tissue, such as skin or other animal tissue, that has been treated with a laser (e.g. at a minimally coagulating setting) to produce voids/channels/grooves in the tissue. It has been surprisingly discovered that voids having a minimal coagulation zone around the ablation void provide a receptive environment into which live cells are readily delivered (without the need for injections into the target tissue) and can therefore survive and persist.

### CROSS REFERENCE TO RELATED APPLICATIONS

This application claims the benefit of the earlier filing date of U.S. Provisional Application No. 61/593,193, filed on Jan. 31, 2012, and U.S. Provisional Application No. 61/716,426, filed on Oct. 19, 2012. The entire content of these prior applications is incorporated herein by reference.

### FIELD

This disclosure relates to methods of implanting or delivering live animal cells into live tissue, for instance into or under the epidermis, the surface of an organ, or otherwise to a specific desired location. Further, this disclosure relates to use of electromagnetic radiation (EMR) to produce voids in tissue into which live animal cells are delivered.

### PARTIES TO JOINT RESEARCH AGREEMENT

This invention was developed subject to a Joint Research Agreement between Dr. David McDaniel and Palomar Medical Technologies, Inc.

### BACKGROUND

Microporation involves the creation of micropores or microchannels in the skin which can be exploited to transport water soluble molecules and macromolecules into and under the skin. Technologies to create microchannels in the skin include mechanical microneedles (Bariya et al., J. Pharm. Pharmaco., 64:1-29, 2011), thermal or radiofrequency ablation and laser ablation (Banga, Expert Opinion on Drug Delivery, 6(4):343-354, 2009). Though there are numerous transdermal delivery systems currently available on the market, the market still remains limited to a narrow range of drugs (Paudel et al., Ther Deliv. 1(1):109-131, 2010).

Separately, methods have been developed to inject various types of autologous or heterologous living cells into tissue (e.g., intradermal injection) for the repair or treatment of, for instance, scars (including acne scars and hypertrophic scars), injuries, wrinkles and other signs of aging, cellulite, wounds, burns, breast and other soft tissue deficiencies, urological structure injuries or deficiencies, diabetes, repair of muscles, vocal cord defects or damage, periodontal disease and disorders, hernias, gastroesophageal reflux damage, tendon and ligament repair, skin pigment modification, hair replacement, and so forth. See, for instance, U.S. patent publications 2011/0110898, 2011/0274665, 2009/0130066, 2008/0311089, 2007/0207131, 2007/0154462, 2007/0154461, 2006/0039896, 2005/0271633, 2005/0186149, 2003/0228286, and 2002/0197241; as well as U.S. Pat. Nos. 5,591,444, 5,660,850, 5,665,372, and 5,858,390. However, there are disadvantages and limitations inherent in applying such cells and cell preparations via needle injection.

### SUMMARY OF CERTAIN EMBODIMENTS

Described herein is the discovery that live cells, such as collagen producing cells (e.g., fibroblasts) or other medically, cosmeceutically, and cosmetically useful cells, can be delivered into (and maintained within) animal tissue by application of the cells to the surface of animal tissue that has been treated with a laser to produce voids in the tissue.

In view of this discovery, there are provided herein methods of delivering live animal cells into tissue, the method comprising ablating a portion of the (target) tissue with electromagnetic radiation to form at least one but more often a plurality of voids (e.g., channels or grooves) extending to a depth below a surface of the tissue; and delivering at least one live animal cell into the void(s). In examples of this embodiment, delivering live animal cell(s) into the one or more voids comprises applying a composition comprising the cell onto the surface of the tissue.

In examples of the described methods of delivery of at least one live animal cell into tissue, the tissue is ablated with electromagnetic radiation, for instance using a so-called fractional laser treatment. By way of example, such methods employ electromagnetic radiation (EMR) having one or more wavelengths of between approximately 1,850 to 100,000 nanometers and with pulse widths of between approximately 1 femtosecond (1×10⁻¹⁵ s) to 10 milliseconds (10×10⁻³ s) with fluence in the range of from approximately 1 J/cm² to 300 J/cm². In other examples, the tissue is ablated with electromagnetic radiation having one or more wavelengths of between approximately 2,200 to 5,000 nanometers. In still other examples, the tissue is ablated with electromagnetic radiation having one or more wavelengths of between approximately 190 to 320 nanometers with fluence in the range of from 1 J/cm² to 300 J/cm². Optionally, conditions selected for ablating portions of the tissue minimize the coagulation zone of tissue damage, for instance by keeping the coagulation zone to a relatively small diameter surrounding the ablated void.

Also described are methods of generating and employing voids of various depths, opening width/diameter, and other characteristics. By way of example, the size of a void may be tailored to accommodate the cell type(s) to be delivered into the target tissue Likewise, the depth of the voids can be tuned to result in placement of cells to desired depth(s) within the target tissue (though it is acknowledged that cells may be placed and will implant somewhat randomly along the depth of the void, and not only at the very bottom). In various embodiments at least a portion of the voids extend to a depth in a range of approximately 0.1 μm to 10 mm. Other depth ranges are specifically contemplated, and the depth selected may be influenced by the type and potentially location of tissue being treated, the type of cell(s) being delivered, and/or both.

Methods involving various target tissues and various types of cells are described herein.

The foregoing and other features and advantages of the invention will become more apparent from the following detailed description, which proceeds with reference to the accompanying figures.

### BRIEF DESCRIPTION OF THE DRAWINGS

FIG. 1 shows human skin explants marked with a Devon® 160-R Skin Marker using a plastic grid template (approximately 0.75 inch squares) to separate each treatment zone, pre- and post-laser treatment and/or application of human skin fibroblasts (HSFs). Specific treatments are detailed in Table 1, below.

FIG. 2A is a series of micrographs showing laser treated human skin explant cross sections (2A, 2D, 2G, 2J, 2M, 2P) as well as fluorescence images of HSF cells applied to the explants by injection or topically after laser treatment (2B & C, 2E & F, 2H & I, 2K & L, 2N & O, 2Q & R). The laser treatments are Palomar STARLUX® 500 Erbium:YAG 2940 nm, green optic, coagulating setting (FIG. A-C; laser setting 24-3-18, which corresponds to 24 mJ/mB, 3 ms, 18 mJ/mB long pulse); Palomar STARLUX® 500 Erbium:YAG 2940 nm, green optic, minimally coagulating setting (FIG. 2D-F); FRAXEL® Thulium:YAG 1927 nm (FIG. 2G-I); FRAXEL® Erbium:YAG 1550 nm (FIG. 2J-L); PALOVIA® 1410 nm (FIG. 2M-O); and FRAXEL® CO₂ 10,600 nm (FIG. 2P-R), as discussed more fully in Example 1. Cells observed in these images are quantified in Tables 3 (topically applied HSF) and 4 (injected HSF).

FIGS. 3A and 3B are graphs showing the absolute number of viable fibroblast cells in laser voids/holes (FIG. 3A) or injected into (FIG. 3B) human skin explants.

FIG. 4 shows five photographs of a human subject's forearm after treatment with a Palomar StarLux 500 2940 nm laser. Two treatments (24 mJ, 850 micron depth, 2% density and 180 MTZ/cm² (single pulse) on the left in each panel; and 12 mJ, 850 micron depth, 2% density and 180 MTZ/cm² (double pulse) on the right in each panel) are shown immediately post treatment, as well as 24, 48, 72, and 96 hours later.

FIG. 5 shows human skin explants marked with a Devon® 160-R Skin Marker using three separate plastic grid templates to ensure no overlap with topically applied cells (approximately 0.75 inch squares) to separate each treatment zone, pre- and post-laser treatment and/or application of human skin fibroblasts (HSFs). Specific treatments are detailed in Tables 6-8, below.

FIGS. 6A and 6B show human skin explant tissue treated with a Palomar STARLUX® Erbium:YAG 2940 nm Groove Optic laser (5 mJ, 120 micron depth, 7% density; setting 5-0-0) prior to topical application of live human fibroblasts. FIG. 6A shows a groove void in cross section, in which fibroblast cells can be clearly seen. FIG. 6B shows a similar groove void under fluorescence imaging, clearly illustrating the labeled fibroblasts.

FIGS. 7A and 7B show human skin explant tissue treated with a Palomar STARLUX® Erbium:YAG 2940 nm Blue Optic laser (9 mJ, 300 micron depth, 4% density; setting 9-0-0) prior to topical application of live human fibroblasts. FIG. 7A shows a drilled void in cross section, in which fibroblast cells can be clearly seen. FIG. 7B shows a similar void under fluorescence imaging, clearly illustrating the labeled fibroblasts.

FIGS. 8A and 8B illustrate a representative positive pressure device (FIG. 8A) and its use in cell application (FIG. 8B).

### DETAILED DESCRIPTION

Unless otherwise noted, technical terms are used according to conventional usage. In order to facilitate review of the various embodiments of the invention, the following explanations of specific terms are provided:

Unless otherwise explained, all technical and scientific terms used herein have the same meaning as commonly understood by one of ordinary skill in the art to which this invention belongs. The word “or” is intended to include “and” unless the context clearly indicates otherwise. Hence “comprising A or B” means including A, or B, or A and B. That is, the word “or” is used in the inclusive sense of “and/or” and not the exclusive sense of “either/or.”

As used herein, the recitation of a numerical range for a variable is intended to convey that the embodiments may be practiced using any of the values within that range, including the bounds of the range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. Finally, the variable can take multiple values in the range, including any sub-range of values within the cited range.

Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### Overview of Several Embodiments

Provided herein in a first embodiment is a method of delivery of at least one live animal cell into tissue, the method comprising ablating a portion of the (target) tissue with electromagnetic radiation to form at least one but more often a plurality of voids (e.g., channels or grooves) extending to a depth below a surface of the tissue; and delivering a live animal cell (or more than one) into one or more of the voids. In examples of this embodiment, delivering live animal cell(s) into the one or more voids comprises applying a composition comprising the cell onto the surface of the tissue, which may be topical application if the target tissue is covered by skin. Optionally, delivering the live animal cell comprises applying positive pressure to the surface of the tissue concurrently with or after applying the composition.

By way of example in various embodiments of the described methods, conditions selected for ablating the portion of the tissue minimize the coagulation zone of tissue damage, for instance by keeping the coagulation zone to a relatively small diameter. Examples are described herein.

In various embodiments at least a portion of the voids extend to a depth in a range of approximately 0.1 μm to 10 mm. Other depth ranges are specifically contemplated, and the depth selected may be influenced by the type and potentially location of tissue being treated, the type of cell(s) being delivered, and/or both.

By way of example, in some described methods at least a portion of the voids extend to a depth of approximately the dermal-epidermal border, to a depth inside the dermal layer, to a depth inside the epidermal layer, to a depth inside the subcutaneous layer, or to a depth deeper than the subcutaneous layer. For instance, in some methods the target tissue is skin tissue and at least some of the voids extend from the surface of the skin tissue to the epidermis. In other methods, the tissue is skin tissue and at least some of the voids extend from the surface of the skin tissue to the dermis. In yet other methods, the tissue is skin tissue and at least some of the voids extend from the surface of the skin tissue to below the dermis.

Also contemplated are methods in which the width or diameter or other measurement of the opening of the void can vary. For instance, in some examples each of the voids (or at least some of the voids) has a width in a range of approximately 0.1 μm to 1 mm. In other examples, each (or at least some) of the voids has a width of approximately 50 μm to 250 μm.

In examples of the described methods of delivery of at least one live animal cell into tissue, the tissue is ablated with electromagnetic radiation, for instance using a so-called fractional laser treatment. By way of example, such methods employ electromagnetic radiation (EMR) having one or more wavelengths of between approximately 1,850 to 100,000 nanometers and with pulse widths of between approximately 1 femtosecond (1×10⁻¹⁵ s) to 10 milliseconds (10×10⁻³ s) with fluence in the range of from approximately 1 J/cm² to 300 J/cm². In other examples, the tissue is ablated with electromagnetic radiation having one or more wavelengths of between approximately 2,200 to 5,000 nanometers. In still other examples, the tissue is ablated with electromagnetic radiation having one or more wavelengths of between approximately 190 to 320 nanometers with fluence in the range of from 1 J/cm² to 300 J/cm².

The tissue that is amenable to treatment with described methods is in some cases human tissue, though the tissue of other animals is also contemplated. Optionally, the human (or other animal) tissue is tissue in a living subject at the time of the ablation and delivery of live cells.

Specifically contemplated are methods in which the live animal cell(s) is autologous to the subject. Optionally, the cell (or collection of cells) is cultured in vitro prior to being applied to the tissue surface.

Cells for use in the described methods can be for instance a fibroblast, an integument cell, an adipocyte, a preadipocyte, a stem cell, an epithelial cell, a retinal cell, an immune function cell, a melanocyte or other pigment cell, a hair follicle cell, a keratinocyte, or a Langerhans cell. Also contemplated are methods of delivery of muscle cells, bone cells, pancreatic cells, cells of a mucosal membrane, chondrocytes, cells of the nervous system, hormone secreting cells, endocrine cells, intestinal cells, and germ cells.

### Cosmetic and Medical Lasers

Electromagnetic radiation, particularly in the form of laser light or other optical radiation, has been used in a variety of cosmetic and medical applications, including uses in dermatology, dentistry, ophthalmology, gynecology, otorhinolaryngology and internal medicine. For most dermatological applications, EMR treatment can be performed with a device that delivers the EMR to the surface of the targeted tissue(s). EMR treatment is typically designed to (a) deliver one or more particular wavelengths (or a particular continuous range of wavelengths) of energy to a tissue to induce a particular chemical reaction, (b) deliver energy to a tissue to cause an increase in temperature, or (c) deliver energy to a tissue to damage or destroy cellular or extracellular structures, such as for skin remodeling.

For skin remodeling, absorption of optical energy by water is widely used in two approaches: ablative skin resurfacing, typically performed with either CO₂ (10.6 μm) or Er:YAG (2.94 μm) lasers, and non-ablative skin remodeling using a combination of deep skin heating with light from Nd:YAG (1.34 μm; 1064 nm), Er:glass (1.56 μm) or diode laser (1.41 μm) and skin surface cooling for selective damage of sub-epidermal tissue. In spite of the differences, in both cases a healing response of the body is initiated as a result of the limited thermal damage, with the final outcome being new collagen formation and modification of the dermal collagen/elastin matrix. These changes result in smoothing out rhytides (wrinkles) and general improvement of skin appearance and texture (often referred to as “skin rejuvenation”).

The principal difference between the two techniques is the region of body where damage is initiated. In the resurfacing approach, the full thickness of the epidermis and a portion of upper dermis are ablated and/or coagulated. In the non-ablative approach, the zone of coagulation is shifted deeper into the tissue, with the epidermis being left intact. In practice, this is achieved by using different wavelengths: very shallow-penetrating ones in the ablative techniques (absorption coefficients of ˜900 cm⁻¹ (10.6 μM) and ˜13,000 cm⁻¹ (2.94 μM) for CO₂ and Er:YAG wavelengths, respectively) and deeper-penetrating ones in the non-ablative techniques (absorption coefficients between 5 and 25 cm⁻¹, for instance through use of Nd:YAG (1.34 μm; 1064 nm), Er:glass (1.56 μm) or diode lasers (1.41 μm). In addition, contact or spray cooling is optionally applied to skin surface in non-ablative techniques, providing thermal protection for the epidermis.

In the cosmetic field for the treatment of various skin conditions, methods and devices have been developed that irradiate or cause damage in a portion of the tissue area and/or volume being treated. These methods and devices have become known as fractional technology. Fractional technology is thought to be a safer method of treatment of skin for cosmetic purposes, because the damage occurs within smaller sub-volumes or islets within the larger tissue volume being treated. Looked at closely, for each laser contact there is a central zone of ablation/vaporization (the void) surrounded by a coagulation zone of irreversible damage/thermal necrosis, which is itself surrounded by a possible zone of reversible thermal injury and then normal tissue. The tissue surrounding the islets is spared from the damage. Because the resulting islets are surrounded by neighboring healthy tissue, the healing process is thorough and fast. Furthermore, it is believed that the surrounding healthy tissue aids in healing and the treatment effects of the damaged tissue.

Examples of devices that have been used to treat the skin during cosmetic procedures such as skin rejuvenation include the Palomar® LuxIR, the Palomar® 1540, 1440 and 2940 Fractional Handpieces, the Reliant Fraxel® SR Laser and similar devices by Lumenis, Alma Lasers, Sciton and many others.

Medical and cosmetic lasers are well known in the art, and are described for instance in published U.S. patent publications 2008/0172047 A1 and 2008/0058783 A1 for instance.

### Methods and Systems for Delivery of Live Cells

It has surprisingly been discovered that live cells can be delivered into voids produced in animal tissue using a laser; cells delivered in the described manner remain viable inside the tissue and are believed to be able to attach and propagate therein. Provided herein are methods of exploiting this discovery, in order to deliver live cells into animal tissue for sustainable tissue enhancement, repair, replacement, healing and the like.

Described method of treatment embodiments involve preparing a target tissue site with a laser in order to create voids in the tissue that are receptive to the live cells to be delivered therein. The laser treatment parameters are selected so as to produce voids of the desired width and depth, so as to produce the desired density of voids in the tissue, and to minimize the thermal coagulation of tissue around the ablation void. Though not previously used to produce tissue voids for receipt of live cells, one of ordinary skill will understand and be familiar with laser parameters that can be selected so as to produce particular characteristics of the resultant voids, such as the extent of the thermal ablation zone, char zone, and/or thermal coagulation zone, as well as the desired depth and diameter to receive specific live cells and deliver them to the desired depth in the target tissue.

The diameter of the voids created by the laser can be between about 50 μm and about 350 μm, between about 70 μm and about 200 μm, or between about 80 μm and about 150 μm.

The depth of the voids can be selected based on (and likely will be influenced by) the part of the body and/or the tissue type to be treated. The voids can extend as deep as needed to reach the desired target site for the live cells to be delivered. Taking skin as an example, the voids can extended into the basal layer of the epidermis, into the dermal-epidermal junction of the skin, or into the dermis of the skin; absolute measurements of these will depend on the specific skin that serves as the target tissue, as it is well known that skin is of different thickness depending on where it is. For instance, epidermis is the thinnest on the eyelids at 0.05 mm and the thickest on the palms and soles at 1.5 mm. The dermis also varies in thickness depending on the location of the skin; it is 0.3 mm on the eyelid and 3.0 mm on the back. Voids made in tissue other than skin, for instance tissue inside the body (reached by way of fiber optic delivered laser pulses, for instance), can be tuned to be of a depth appropriate for that tissue and to permit delivery of cells to the depth in that tissue that is desired for the elected treatment.

The depth of the voids in various embodiments is between about 10 μm and about 4 mm or more (for instance, if the voids extend through the skin and the underlying hypodermis and/or fat, in some instances all the way to superficial bone or cartilage), between about 50 μm and about 3 mm, or between about 10 μm and about 850 μm.

As demonstrated herein, viable delivery into and/or maintenance of cells within laser-drilled voids in live tissue is influenced by the amount of coagulation of tissue around the void. Thus, in various embodiments, it is considered beneficial to minimize the zone of coagulation generated by the laser treatment. While not intending to be limited by any proposed hypothesis, it is believed that coagulated tissue surrounding the void results in a less advantageous environment for the introduced cells to survive. Thus, in certain embodiments the coagulation zone around the laser-generated void (e.g., channel or groove) is minimized, for instance is no more than 0-50 microns thick on average, or no more than 2-30 average microns, no more than 5-20 average microns and in certain embodiments less than 10 or even less than 5 microns thick, on average. It is recognized that the thickness of the coagulation region, similarly to the diameter or width of the void itself, is variable in part due to the heterogeneity of the tissue.

Void diameter and coagulation zone thickness for various laser treatment parameters can readily be characterized using techniques that are recognized in the art. By way of example, the following table provides such statistics for the Palomar StarLux 2940 nm laser using various optics and settings.

### Lux2940 Optic Characteristics Summary in Ex Vivo Porcine Skin

| | | | Damage | | % | |
|---|---|---|---|---|---|---|
| | | Damage | Diameter | | Coverage | |
| | | Depth | (ablation + | | per shot | |
| | | (μm) | coag, μm) | Coag | (@ D/E jct) | |
| Optic | Setting* | +/−15% | +/−15% | Thickness | +/−15% | Notes |
| 300 1.5X | 9-0-0 | 300 | 100 | 5-10 | 4 | |
| (Blue) | 0-3-7 | 215 | 140 | 10-20 | 7.5 | |
| | 0-5-8 | 240 | 120 | 10-20 | 6 | |
| | 9-3-7 | 400 | 120 | 35-50 | 6 | |
| | 9-5-8 | 430 | 150 | 35-50 | 9 | |
| 300 2.5X | 9-0-0 | 115 | 100 | 5-10 | 1.5 | |
| (Red) | 9-3-7 | 200 | 130 | 35-50 | 2.5 | |
| | 0-3-7 | 110 | 110 | 10-20 | 2 | |
| 500 1.5X | 25-0-0 | 850 | 120 | 15-25 | 2 | |
| (Green) | 25-5-24 | 925 | 170 | 35-50 | 4 | |
| 2 mm | 25 J/cm² | 100 | | | 100 | 100 micron |
| optic | | | | | | depth per |
| | | | | | | 25 J/cm² |
| Groove | 5-0-0 | 120 | 90 | ND | 7 | 1350 pitch |
| (Silver) | 5.5-0-0 2S | 270 | 130 | ND | 10 | |
| | 5.5-3-4.5 | 150 | 150 | ND | 10 | |
| | 5.5-3-4.5 2S | 300 | 170 | ND | 20 | |
| | 5.5-5-5 | 200 | 140 | ND | 15 | |
| | 5.5-5-5 2S | 330 | 150 | ND | 24 | |

S = Stacked pulses
*Instrument settings for 2940 nm laser: x-y-z, where X is the mJ/mb @ 250 microsecond (which is the duration of the short pulse), Y is the pulse duration of the long pulse in milliseconds, and Z is the mJ/mb of the long pulse.

Characterization of voids (also referred to as ablation islets, micro-islets, grooves, micro-grooves, etc.) made in tissue using lasers, including parameters for varying the dimensions and other characteristics of such voids, can be found in the art, for instance in U.S. Patent Publication No. 2009/0069741. That publication also provides examples of methods for creating micro-holes and other EMR-generated voids in animal tissue.

In certain embodiments, it is beneficial to match laser-generated void characteristics with the type of cells to be delivered into the target tissue, as well as the tissue into which the cells are to be delivered. For instance, the void diameter (or groove width in some embodiments will be equal to at least 1× the diameter of the cell type to be delivered, at least 1.5× the diameter, at least 2×, 3× or even 5× or more the diameter of the cell type to be delivered into the void. Cell sizes are known in the art for at least some cell types. For instance, fibroblasts once they are contacted into roughly spheres are around 80-100 μm in diameter. Melanocytes have a diameter of around 2 μm. Adipocytes vary in size based on body fat levels such that a person with a normal BMI has adipocytes around 1700 μm² and having a diameter of approximately 46 μm, assuming a roughly spheroid shape (see, e.g., Gealekman et al., Circulation 123(2):186-194, 2011; Yecies et al., Cell Metabolism 14(1):21-32, 2011). For cells that are not previously characterized, standard laboratory techniques can be used to measure their average diameter prior to using them for implantation.

Depending on the desired treatment, it may also be beneficial to place or distribute cells differently or differentially within the target tissue. For instance, fibroblasts being implanted to increase collagen production in skin may be distributed them throughout the dermis, for instance using a target zone of 1-3 mm penetration for the thickness of dermis, and as shallow as 0.1 mm to target the epidermis where it is thin, for instance in old photoaged skin. Alternatively, deliver into the epidermis (rather than through it or transepidermal delivery) would employ less deep delivery, while placement into fat tissue would be deeper, for instance 3 mm or more. In some combination treatments, cells (for instance, cells of different types) are delivered to different depths in the same target tissue.

It various embodiments, it may also be beneficial to tailor laser settings in order to reduce negative tissue responses, and in particular to reduce responses in the target tissue that may reduce the viability of the implanted cells. For instance, it is beneficial to reduce or limit the amount of bleeding caused by the laser treatment (though this must be balanced with the desirable minimization of coagulation of the tissue surrounding the laser-induced void).

It may also be beneficial to reduce elicitation (in the target tissue) of a non-supportive biological environment, with regard to cellular responses and cell signalling. One way to monitor and influence this is to track protein (or gene) expression in response to varying laser treatment, and tailor laser treatment to minimize expression of factors that are not supportive to implantation or growth of the implanted cells.

Depending on the desired clinical or therapeutic application goal, the distance or spacing between the voids may be varied to produce more uniform coverage. Similarly the choice of spacing between voids may be chosen based on the type of cell and its ability to migrate. In the case of melanocyte cells, the spacing selected may be influenced by the goal of obtaining visually pleasing pigmentation.

### Optional Tissue (e.g., Skin) Pretreatment

It may be beneficial to embodiments of the cell delivery systems described herein for the skin to be clean and free of debris (dead skin, dust, numbing cream crystals, etc. . . . ). Such cleaning is beneficially carried out using a cleaning solution and/or procedure that is not toxic to cultured cells to be applied to the skin, so as to prevent any residual cleaning solution from harming the topically applied fibroblasts.

As described herein, testing was performed on examples of common solutions that may be used by physicians for cleansing or numbing the tissue to be treated in the cell delivery procedures, and on coagulation agents used in the cell delivery procedures, in order to determine the effects (if any) of solutions and agents on the cultured cells. Based on the tests reported herein (and summarized in the following paragraph), certain potential treatment compounds are recommended not to be used use due to cytotoxic effects.

The following treatments are disfavored, and considered generally inappropriate for preparing tissue (e.g., skin) for the cell delivery methods provided herein: use of compounds tested and known to be cytotoxic to cultured human skin fibroblasts; incomplete removal of numbing agents and/or excess moisture (water droplets) from the skin prior to laser treatment; application of any substance or composition that impedes laser function and cell penetration (masques, etc. . . . ); omitting to cleanse the skin (as this could be detrimental to the procedure's efficacy, as layers of dead skin, dirt and debris may impede laser function, fibroblast penetration and fibroblast adhesion and may potentially encourage or cause infection).

The current representative treatment protocol provided herein uses a gentle cleanser (for example, a non-cytotoxic cleanser) and an ultrasonic brush to remove the numbing agent. The skin is then allowed to dry, as water on the skin is likely to reduce or block the penetrating power of the laser, resulting in fewer (or no) holes being created (or the holes will have reduced depth). This can lead to fewer topically applied fibroblasts penetrating to the desired location and depth in the selected tissue (e.g., skin).

### Types of Cells/Types of Treatments

Contemplated herein are treatments involving delivery of cells into or through skin tissue, into the mucosa in the mouth or other lining cells, as well as delivery of cells into fat or muscle, and delivery of cells into other tissues. Such treatments include for instance reduction of wrinkles by inserting fibroblasts that can or are producing collagen (see U.S. patent publication No 2007/0154462); replacement of fat cells/adipocytes (see U.S. Patent publication no. 2007/0154461) including for fat grafting and filling in sunken scars, age related fat atrophy, post traumatic fat atrophy, pressure induced fat atrophy; replacement of melanocytes (see e.g. U.S. patent publication No. 2007/0225779, which describes a different method of delivering melanocytes to skin); treatment of hypertrophic scar tissue (see, e.g., U.S. patent publication 2010/0189694); enhancement of sphincter function using delivered muscle cells (see, e.g., U.S. Patent publication no. 2009/0130066); and so forth. In each instance, the herein described methods of generating voids in tissue using a minimally-coagulating laser are exploited to permit delivery into those voids live cells that previously had to be delivered by needle injection.

In other embodiments there are contemplated treatments of tissues other than skin and the tissue proximally under skin. These include for instance treatment of vocal cord tissue defects (see, e.g., U.S. patent publication 2008/0311089), of esophageal damage such as that caused by acid reflux, and treatment of various other conditions (see, e.g., U.S. patent publications 2005/0186149, 2006/0039896, 2005/0271633, 2011/0110898 and 2007/0207131, which also describe various cell types and methods of their production). Thus, specifically contemplated are embodiments where cells are to be delivered inside of an animal's body. In order to prepare such tissue for receipt of the cells, the laser's energy may be delivered using fiber optics; such lasers are well known to those of skill in the art. In such embodiments, the term “topical” as used herein (e.g., to refer to topical application of cells to laser treated tissue) refers non-traditionally to application to the surface of the selected tissue even though that tissue is not covered with skin (and thus is not what might normally be considered “topical”). Optionally, access to the internal organ/tissue may be by way of endoscopy or other surgery.

With the methods and technologies provided herein, there are now enabled methods of repairing internal scarring (e.g., in the throat or esophagus, in joint or other cartilage, in the eye), replacement of defective cells (including, but not limited to, replacement of pancreatic cells such as Islets of Langerhans cells), and localized gene therapy through specific placement of engineered cells into specific target tissues.

It will be recognized by one of ordinary skill in the art that laser treatments are usually carried out over a period of time—for instance, a first laser application followed by a period of time that may be several days or weeks which is then followed by another (similar or different) laser treatment. More than two treatments may be applied. The technology described herein is similar, in part because the voids used for cell delivery are generated using a laser/lasers and thus the target tissue benefits from periodic abstinence from laser exposure, and also because serial cell implantation can be beneficial in some instances.

Though generally described herein in the context of single-cell-type treatments, also contemplated are treatments that employ a mixture of cell types—including different sources of cells, cells that are differently differentiated, different cells that are implanted into the tissue to different depths, and so forth. The cells may be applied to the tissue as mixtures or sequentially, and if sequentially they may optionally be applied over a time course of days or weeks for instance.

### Sources, Growth and Production of Cells for Delivery

Though cells for use in the described methods may be from almost any source, it is beneficial that the cells be autologous to the recipient in order to minimize rejection effects. One non-limiting example of autologous cells is the LaViv® (axficel-T) autologous fibroblast system from Fibrocell (see, e.g., U.S. Patent publication no. 2011/0274665, as well as U.S. Pat. Nos. 5,591,444, 5,660,850, 5,665,372, and 5,858,390), which type of cell system is specifically contemplated for use with the implantation methods described herein.

Cells for use in the methods described herein can be isolated from harvested animal tissue. Though the examples and much of the description herein are provided in the context of human treatment, it is specifically contemplated that the provided technologies and methods can be used in veterinary systems as well.

In general, methods of isolation of cells include not only harvesting a tissue specimen, but also processing the specimen so that the cells contained therein are substantially dissociated into single cells rather than grouped as cell clusters. Dissociating the cells into single cell components can be accomplished by any method known in the art; e.g., by mechanical (filtering) or enzymatic means. Further, the isolating step includes combining the cell-containing specimen with a cell culture medium comprising factors that stimulate cell growth without differentiation. The specimen-medium mixture is then cultured for a few up to many cell passages.

Appropriate culture medium is described and well known in the art. For example, stem cells can be cultured in serum free DMEM/high-glucose and F12 media (mixed 1:1), and supplemented with N2 and B27 solutions and growth factors. The media can be used in the absence of a feeder layer, but in the presence of a matrix coated tissue culture dish. The matrix can be selected from fibronectin, collagen, laminin, and combinations thereof. Equivalent alternative media and nutrients can also be used. Culture conditions can be optimized using methods known in the art.

Cells can be cultured in suspension or on a fixed substrate. For example, the cells can be grown on a hydrogel, such as a peptide hydrogel, using well known techniques. Alternatively, the cells can be propagated on tissue culture plates or in suspension cultures. Cell suspensions can be seeded in any receptacle capable of sustaining cells, particularly culture flasks, cultures plates, or roller bottles. The cells can be grown on tissue culture plates, and can be cultured at high cell density.

Conditions for culturing human and other animal cells in vitro are well known to those of ordinary skill, and are usually close to physiological conditions. The pH of the culture medium is usually close to physiological pH, for instance between pH 6-8, between about pH 7 to 7.8, or around pH 7.4. Physiological temperatures (for human cells) range between about 30° C. to 40° C.; other temperatures may be appropriate for the culturing of cells from other animals. Human cells are preferably cultured at temperatures between about 32° C. to about 38° C., for instance between about 35° C. to about 37° C. Cells may be cultured for 3-30 days, for instance at least about seven days, at least 10 days, or at least about 14 days. Cells can be cultured substantially longer. They can also be frozen using recognized methods such as cryopreservation, and thawed and used as needed.

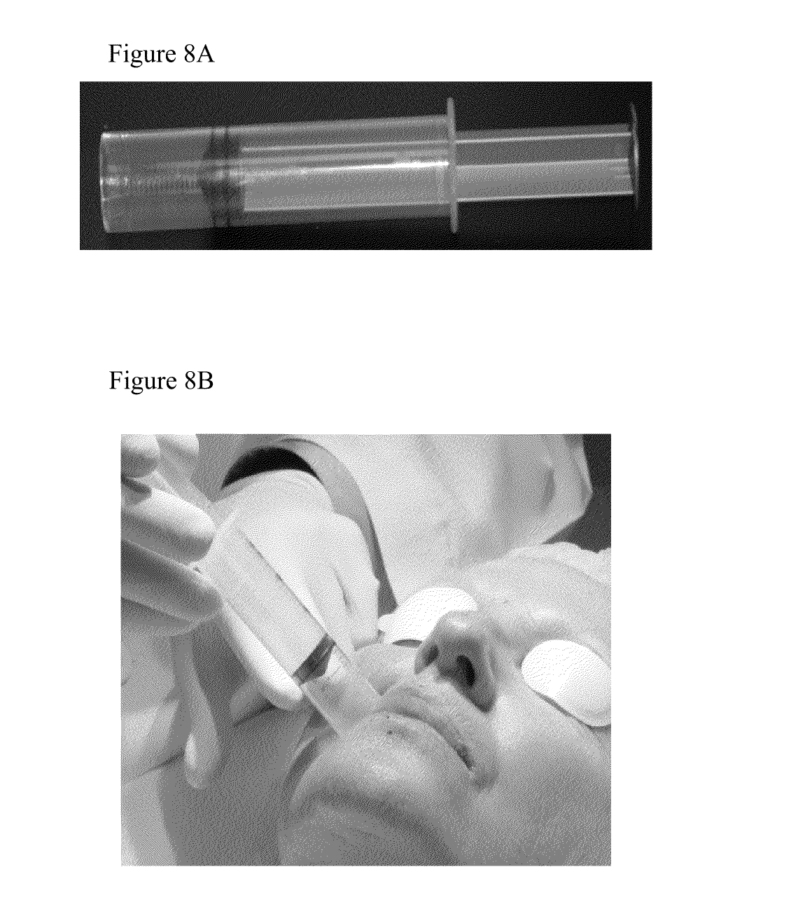

Live cells can be delivered to laser treated tissue using various methods that do not rely on injection, including direct and passive surface application, surface application with some active assistive means. Representative examples of assistive means include, but are not limited to, absorption, vibration, other mechanical stimulation (such as massaging of the tissue), applying positive pressure (e.g., direct pressure on the tissue (for instance using physical contact, gas pressure, liquid pressure, and so forth; see FIG. 8 for instance), placing the subject or tissue into a hyperbaric chamber, a mini-hyperbaric chamber device that is placed on a region of the skin and sealed), applying electrical or magnetic fields, application of a jet spray and application of acoustic energy such as ultrasound)

Following delivery of cells to a laser treated tissue, an occlusive bandage, or other barrier, can be fixed to the tissue to retain the cells within the voids and/or to reduce or prevent vapor loss from the tissue. Beneficially, any such barrier is substantially non-toxic to the implanted cells. Optionally, the barrier may contain or be impregnated with one or more positive factors that encourage implantation and/or growth of the implanted cells, such as growth factors and the like.

### Optional Accompanying Treatments

The live cells can optionally be delivered with other components, for instance compounds, mixtures, compositions, suspensions, and may include components in different phases, such as small solid particulates in a liquid.

Fibroblast cells that are being applied for improvement of collagen for or formation may optionally be implanted along with (or in series with) structural matrices such as collagen-based matrices as are recognized in the art, and optionally a collagen production stimulant. See, for instance, Varani et al. (Am J Pathol 168(6):1861-1868, 2006), which describes the involvement of collagen and aged fibroblast cells in skin structure with age. Also contemplated is application of hyaluronic acid (HA), elastin, collagen, etc.

For scar treatment it may be beneficial to include or pre-treat the target tissue with a collagen-degrading (e.g., MMP) or reformatting composition (e.g., collagenase), or a stimulant for collagen production.

Also contemplated is inclusion of compounds (e.g., biologically active compounds) that support or encourage implantation or maintenance or growth or reproduction of the implanting cells and/or the cellular environment within the induced voids. Such compounds include but are not limited to antioxidants, growth factors (which may optionally be selected to be specific for or particularly tailored to the implanting cells), addition of critical co-factors, enzymes, co-enzymes, chemokines, cytokines, extracellular matrix and/or adhesion molecules, siRNA molecules, antibiotics, recombinant DNA/retrovirus, microRNAs, and so forth.

By way of example, compositions that are applied with live cells as described herein may include compositions and mixtures injected with cells using previously taught technologies. For instance, U.S. Patent publication no. 2011/0274665 describes in detail various compositions for use with injectable autologous fibroblasts, which can readily be adopted for use with the methods and technologies described herein. Other such compositions will be known to those of skill in the art.

### Representative Treatment Protocol

Without intending to be bound by any one particular protocol or method, the following is provided as an example “same day” procedure for treatment of the face or other skin section using a method described herein (including optional elements).
1. Patient arrives at office with no makeup or skin care lotion on face and/or areas to be treated.
2. Patient fills out and signs consent form, and optionally photo release.
3. Give patient one ibuprofen (or equivalent mild anti-inflammatory/pain killer) only.
4. Take “pre-treatment” photos if not done at Pre-op visit.
5. Cleanse patient's face using Clarisonic® sonic skin cleansing brush (or equivalent) on low setting using the SkinCeuticals® Gentle cleanser (or equivalent, such as a non-cytotoxic cleanser) using the sensitive brush.
6. Optionally, perform one treatment of GentleWaves® skin treatment yellow to all areas to be treated (GentleWaves® is a LED (light emitting diode) Photomodulation System).
7. Apply 30% lidocaine ointment (do NOT use cream base, use only petrolatum base) for numbing, 30 minutes.
8. Cleanse face using Clarisonic® skin cleansing brush on gentle setting using the SkinCeuticals® Gentle Cleanser or equivalent (such as a non-cytotoxic cleanser).
9. Make sure all lidocaine ointment is off before proceeding.
10. Mark the face with the patient sitting up; use a washable style marker, such as Crayola® washable markers.
11. Insert eye shields (if treating under eyes) or otherwise use microderm “stickies” if full face treatment, or use gauze pads.
12. Treat the skin using the 2940 nm Palomar laser and Blue handpiece on setting 5/0/0 with a repetition rate of about five around eyes and typically about three elsewhere, then switch to Green handpiece at 22/0/0. Holding the laser sideways for better vision and stability, treat the areas in a tile-laying manner with 0-10% overlap and one pass. Stretch skin where needed to obtain complete coverage.
13. Divide the face into thirds and treat, for instance, in the following order: 1. Left side of face to midline from jaw to brow; 2. Right side of face to midline from jaw to brow; and 3. Full forehead. Cells are applied to skin after each section is finished, Do NOT wait to apply until the entire face is treated. Clean the optics after each section.
14. While treating, keep the vacuum close to the skin and wipe the optical window frequently.
15. It is acceptable to use ice on the skin surface before applying cells; however, do not apply ice after cells have been applied.
16. To prevent bleeding: Blot (but do not wipe!) with dry gauze. If needed, 1:100,000 epinephrine may be applied to the skin, for instance using a cotton swab to only the spot(s) that is bleeding.
17. Apply autologous cells topically to the treated area via 1.0 cc syringe with needle removed, and gently massage in with sterile gloved hands to each zone/segment treated (in case of face, one third of the skin in the order in which it was treated); then apply second application of cells 1-2 minutes after first application of cells. Estimated volume for full face is approximately 1 cc per coating. Optionally, apply one coat of patient's “conditioned media extract” and invert the cells gently before using them for treatment. Optionally: Treat cell-covered areas using positive pressure, for instance using a device that applies gentle positive gas pressure such as the “positive pressure syringe” device (FIG. 8A), to encourage cells into perforations in the skin.
18. Optionally: Apply Skin Resuscitation Factor™ (SRF™) anti-inflammatory ointment 1-2 minutes after the second application of cells. If doing treatment in stages of anatomic units, be careful to not get SRF™ ointment on the yet-to-be-treated areas; leave a small margin not covered with SRF™ and overlap later. If SRF™ ointment not used, apply plain petrolatum in its place
19. Review aftercare instructions with patient; set up follow up appointments and confirm patient has aftercare supplies. Typically, follow up appointments occur at 3 days, one week, two weeks and one month and six months post treatment.
20. Take immediate post treatment photos (optional).

The following examples are provided to illustrate certain particular features and/or embodiments. These examples should not be construed to limit the invention to the particular features or embodiments described.

### EXAMPLES

### Example 1

### Delivery of Live Human Skin Fibroblasts to Skin Tissue

This example describes methods of preparing cells and tissue for live cell delivery, including characterization of laser treatment conditions conducive to delivery of live fibroblast cells into human skin tissue explants.

### Materials & Methods

### Cell Culture & Fluorescent Labelling

AG07999 human skin fibroblasts (HSF; Coriell, Camden, N.J.) were grown to confluence in minimal essential media (MEM; Gibco, Grand Island, N.Y.) supplemented with 10% FBS (Hyclone, Logan, Utah), 20 mM L-glutamine, and 20 mM Glutamax™ (Gibco). Cells were maintained in an incubator at 37° C. and 5% CO₂ and serially passaged with trypsin digestion at a 1:3 ratio as needed.

Prior to experimental use, HSF were labelled with 2.5 μM CFDA-SE (carboxyfluorescein diacetate, succinimidyl ester; Molecular Probes, Eugene, Oreg.) according to manufacturer's protocols for non-adherent cell lines. Although AG07999 is an adherent cell line, the volume of cells required for this experiment made it technically easier to label the cells according to the manufacturer's protocol for non-adherent cells. Briefly, cells were collected by trypsin digestion and pelleted by centrifugation, then resuspended in excess pre-warmed 2.5 μM CFDA-SE prepared in PBS (phosphate buffered saline). The CFDA-SE compound is inactive and cell permeable until it enters cells, where it is metabolically cleaved and activated. The resulting fluorescent compound is cell-impermeable and is therefore retained in the cells as long as they remain viable. This compound has an excitation maximum at 488 nm and peak emission at 517 nm; it is therefore visible with most filters compatible with FITC (fluorescein isothiocyanate) or GFP/EGFP (green fluorescent protein/enhanced GFP). HSF were labeled with the CFDA-SE for 15 min, then pelleted by centrifugation and resuspended in fresh pre-warmed PBS. HSF were then incubated an additional 30 min and were pelleted, resuspended in fresh PBS, and counted via haemocytometer. Cell density was adjusted to 1.63×10⁷ cells/mL, and samples were maintained on ice until use.

### Skin Explants

Human skin was obtained from an abdominoplasty surgery patient. Following surgical excision, tissues were maintained in sterile saline on ice for transport. On arrival, skin was de-fatted, rinsed through two changes of PBS, and maintained in sterile PBS on ice until use. Prior to use, skin was pinned to a dissecting tray, allowed to warm to room temperature, and patted dry.

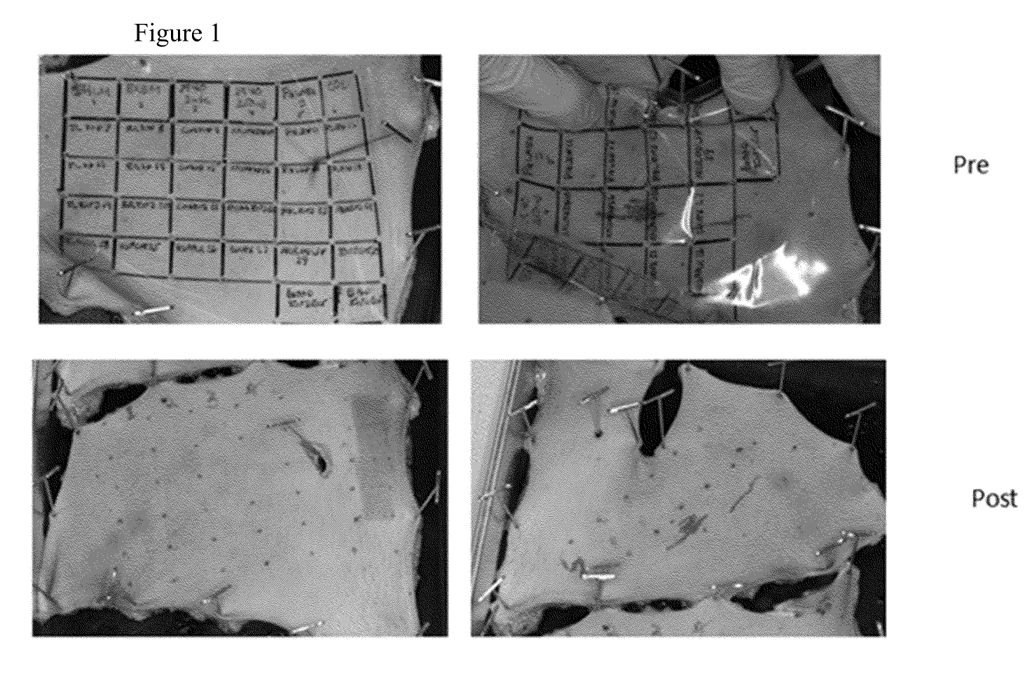

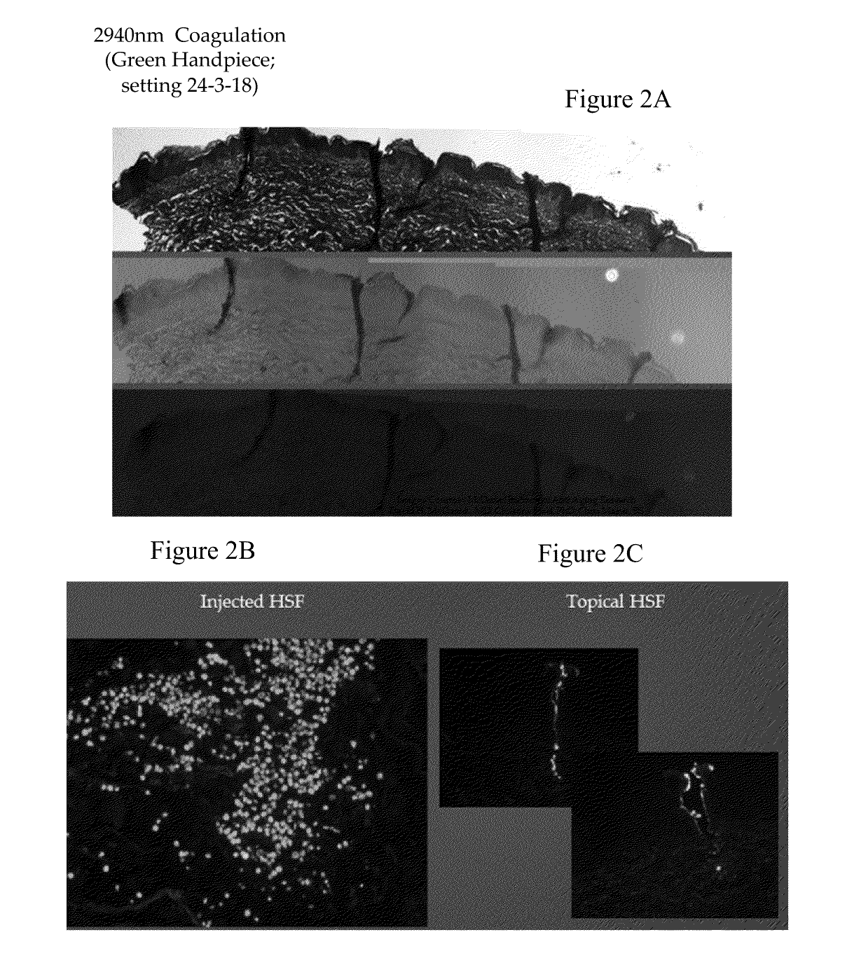

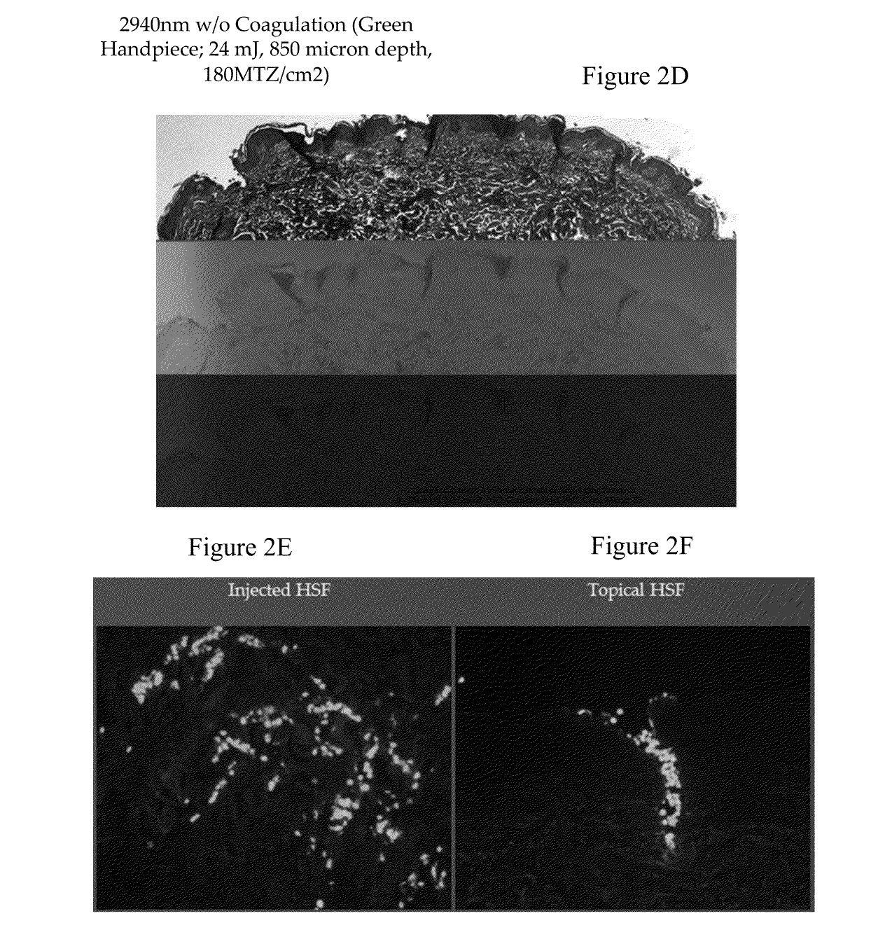

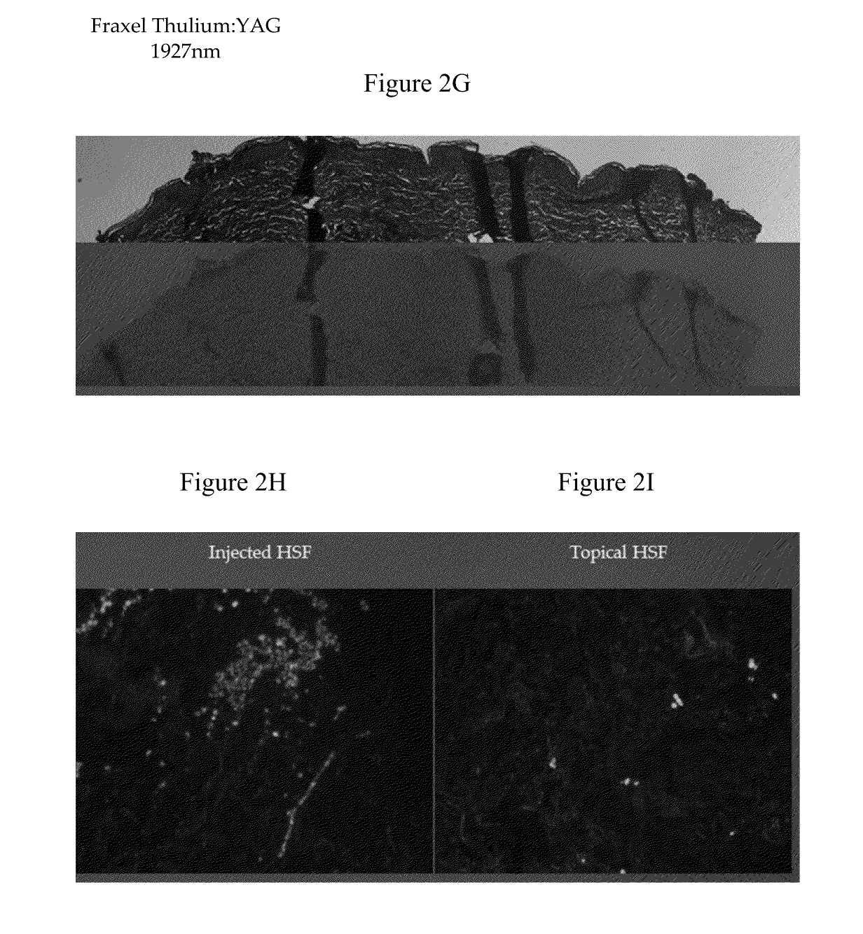

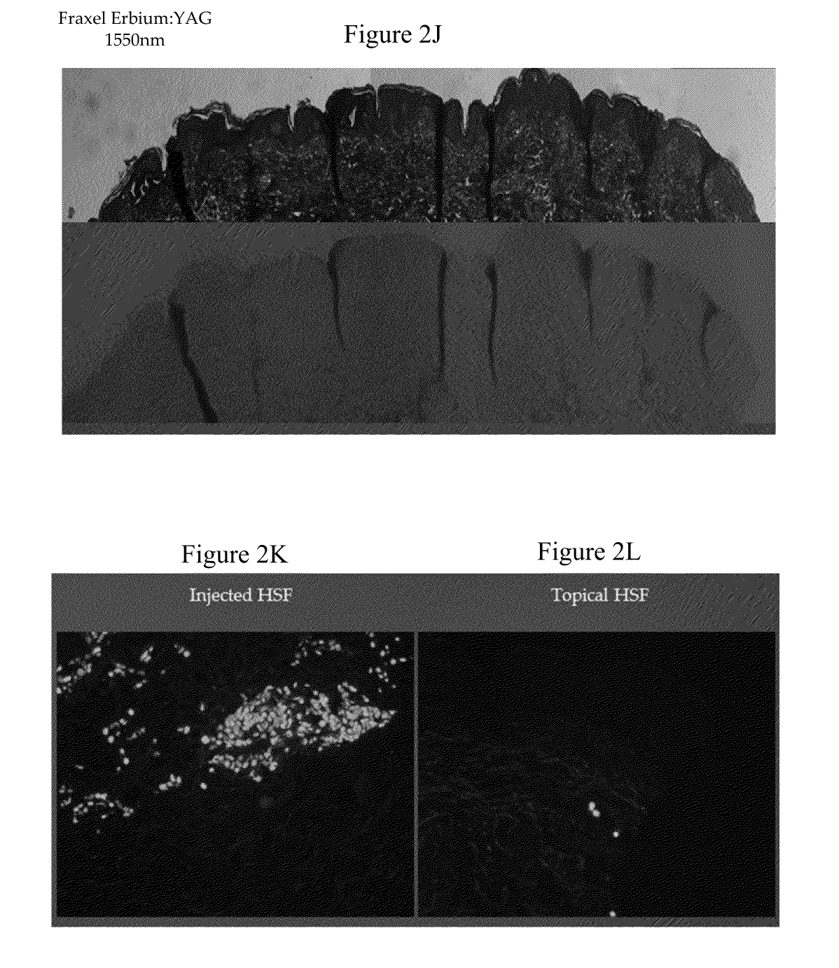

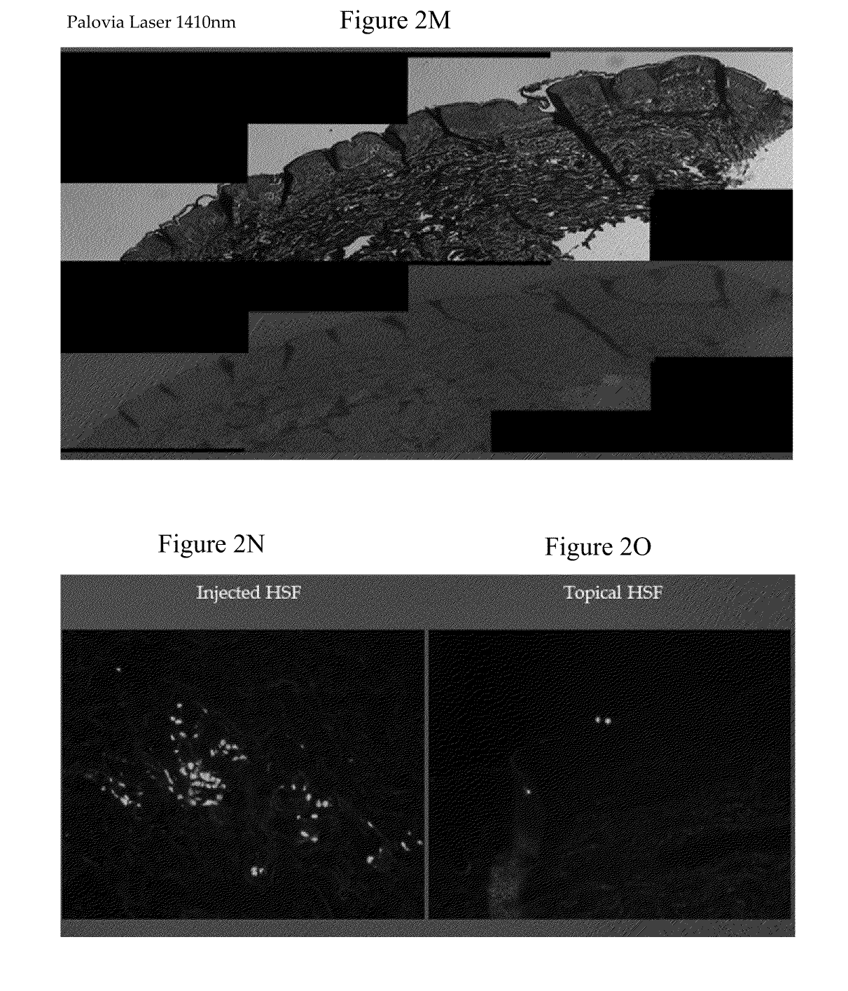

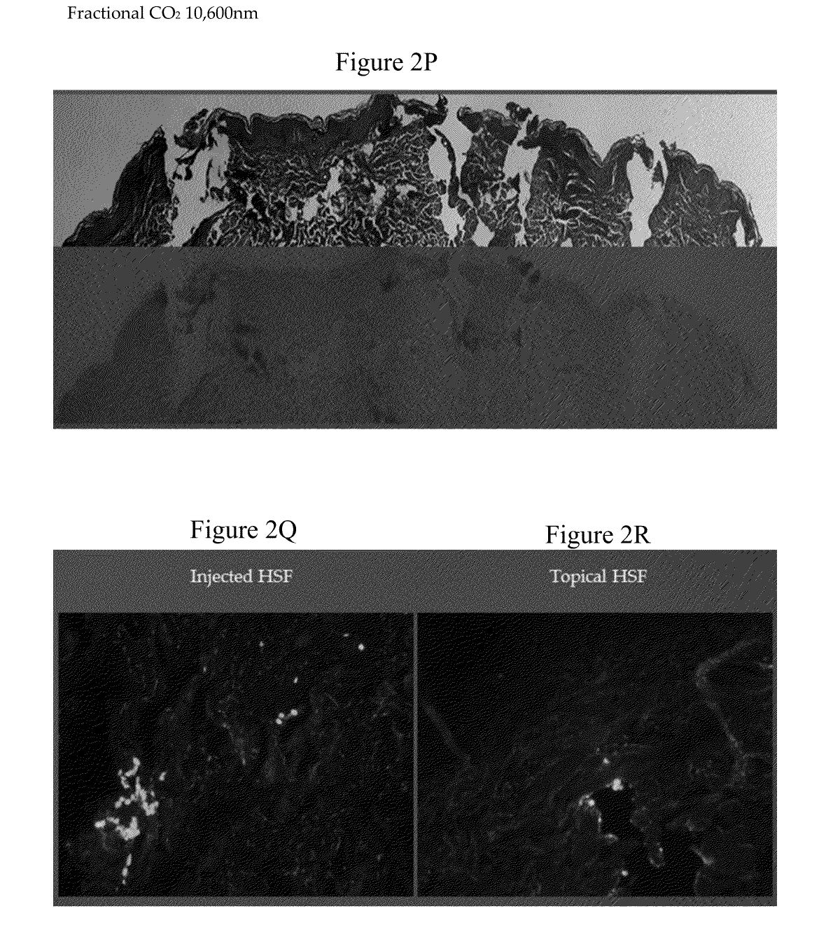

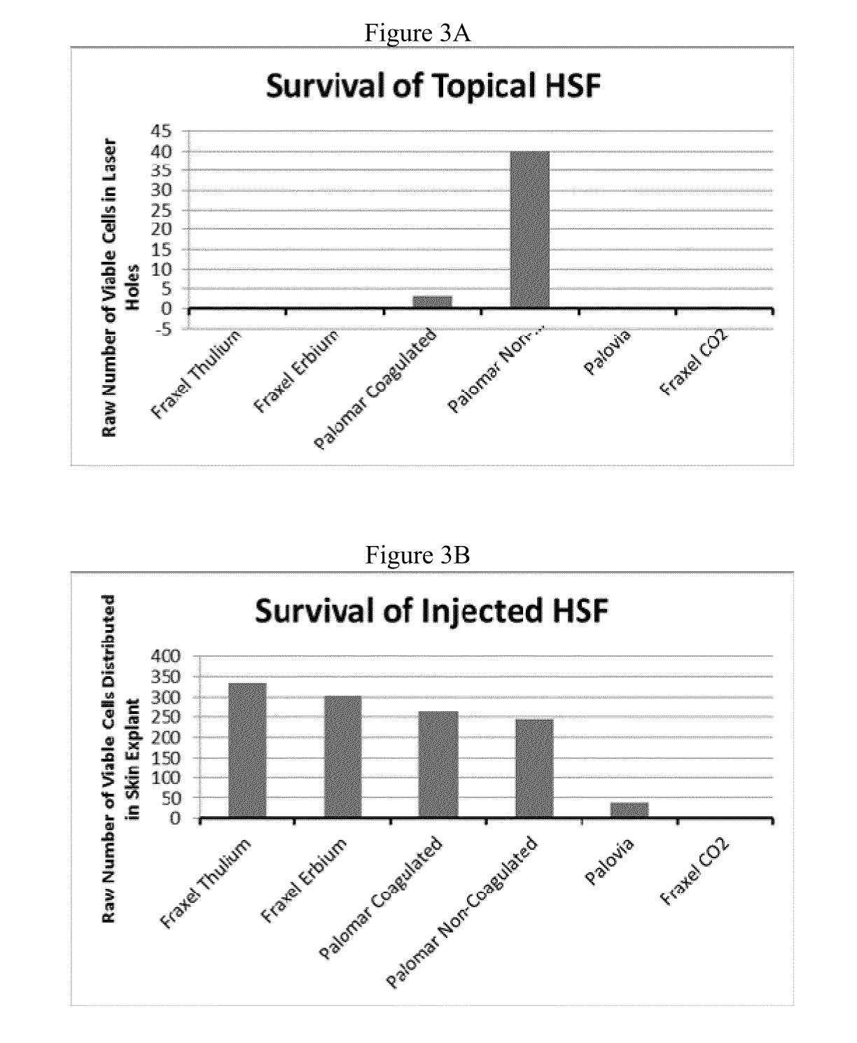

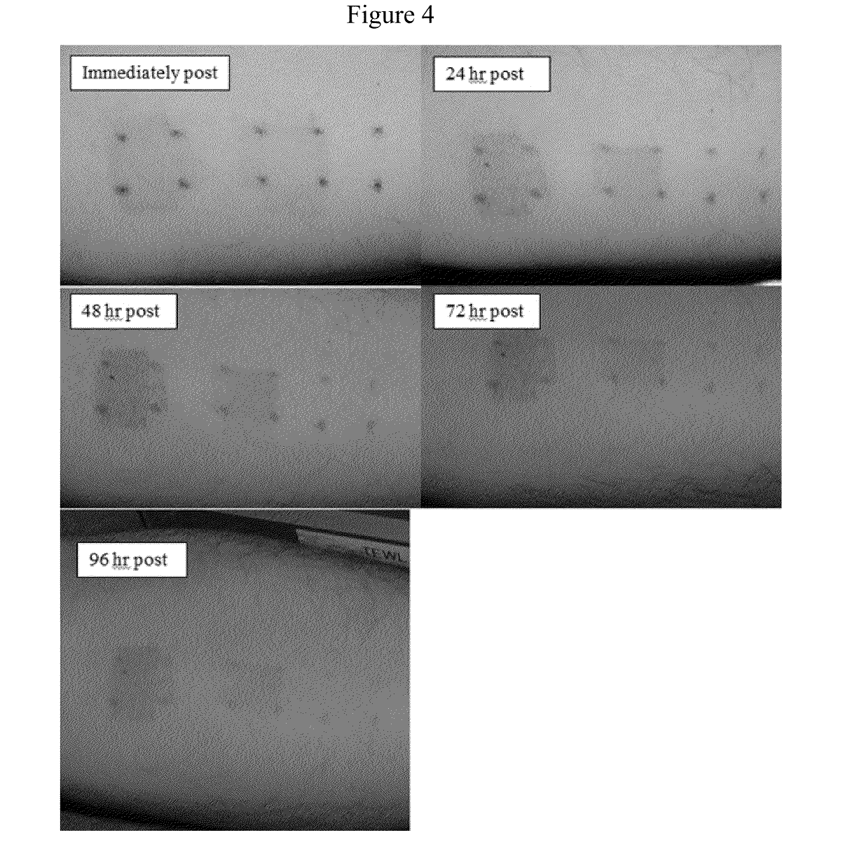

Skin explants were marked with a Devon® 160-R Skin Marker using a plastic grid template (approximately 0.75 inch squares) to separate each treatment zone. The grid (shown in Table 1, below) was designed to have identical laser settings performed in vertical columns and each laser treatment condition (laser alone, laser+topical Human Skin Fibroblast application, Laser+15 μm red fluorescent microspheres, and laser+injected HSF). After all individual grids were marked, the template was removed and each column numbered from left to right. Each laser was then applied to each respective grid for each respective setting. See FIG. 1.

TABLE 1
Laser Treatment Experimental Layout.

| | | | Palomar | Palomar | | |
|---|---|---|---|---|---|---|
| | Fraxel | Fraxel | Erbium: | Erbium: | | |
| | Thulium: | Erbium: | YAG | YAG min- | Palovia ® | |
| Condition | YAG | YAG | Coagulated | Coagulated | Diode | Fraxel CO₂ |
| Laser | TL 1 | ERB 2 | COA 3 | NCOA 4 | PAL 5 | FCO 6 |
| Laser + | TLTOP 7 | ERBTOP 8 | COATOP 9 | NCOATOP | PALTOP 11 | FCOTOP |
| Topical HSF | | | | 10 | | 12 |
| Laser + | TLSP 13 | ERBSP 14 | COASP 15 | NCOASP 16 | PALSP 17 | FCOSP 18 |
| Spheres | | | | | | |
| Laser + | TLINJ 19 | ERBINJ 20 | COAINJ 21 | NCOAINJ 22 | PALINJ 23 | FCOINJ 24 |
| Injected HSF | | | | | | |
| Addt'l | TLEGCG | HSFCNT | HSFFLLR | UNTX 27 | MCRNDLSP | INJCNT 30 |
| Controls | 28 | 25 | 26 | | 29 | |
| Injection | | | | | Good Inj | Bad Inj |
| Technique | | | | | Tech 40 | Tech 41 |

The experimental layout and design template is shown in Table 1, including all additional controls. Fluorescent microspheres (15 μm diameter, Invitrogen, Grand Island, N.Y.) were topically applied to some samples. However, these microspheres are polystyrene and are believed to have dissolved during histological processing. Thus, they are not visible in final samples (except for the injected control sample).

Additional controls included injected CFDA-SE labelled HSF. These groups served as the positive controls, to demonstrate that the fluorescently-labelled cells persisted in the ex vivo skin, had approximately normal morphology, and remained fluorescent following histological processing. Massons' Trichrome staining was observed to quench or destroy fluorescence from CFDA-SE; thus, unstained slides were imaged.

### Laser Devices & Parameters

Six different laser treatment parameters were evaluated. These devices and the parameters and instrument settings used are summarized in Table 1. Reliant FRAXEL® lasers (Thulium:YAG 1927 nm and Erbium: YAG 1550 nm, as FRAXEL® Dual; and FRAXEL® CO₂ 10,600 nm) were purchased from Solta Medical, Inc. (Hayward, Calif.). The Palomar STARLUX® 500 Erbium:YAG (2940 nm) laser was kindly loaned from Palomar for the purposes of this study (Palomar Medical Technologies Inc., Burlington, Mass.). The Palomar PALOVIA® (1410 nm) and Palomar YAG 5 (Q-Switched Nd:YAG laser) lasers were purchased from Palomar (Palomar Medical Technologies Inc., Burlington, Mass.).

TABLE 2
Laser Device Parameters and Instrument Settings.

| | | | | Palomar | | |
|---|---|---|---|---|---|---|
| | | | Palomar | Erbium: | | |
| | Fraxel | Fraxel | Erbium: | YAG | | |
| | Thulium: | Erbium: | YAG | minimally | Palovia ® | Fraxel ® |
| Settings | YAG | YAG | Coagulated | Coagulated | Diode | CO₂ |
| Wavelength | 1927 nm | 1550 nm | 2940 nm | 2940 nm | 1410 nm | 10,600 nm |
| Energy | 15 mJ | 40 mJ | 24 mJ/mB | 24 mJ/mB | 15 mJ | 70 mJ |
| | | | 18 mJ/mB | 0 mJ/mB | | |
| Depth | 199 μm | 1120 μm | 850 μm | 850 μm | n/a | 1579 μm |
| Density | 35% (4) | 32% (11) | 2% | 2% | n/a | 35% (9) |
| MTZ/cm² | 80 (4) | 84 (8) | 180 | 180 | n/a | n/a (4) |
| Instrument | 15-4-4 | 40-11-8 | 24-3-18* | 24-0-0* | Level 3 | 70-9-4 |
| Settings | | | | | | |

mB: “microbeams” as per manufacturer's specifications.
*Instrument settings for 2940 nm laser: x-y-z, where X is the mJ/mb @ 250 microsecond (which is the duration of the short pulse), Y is the pulse duration of the long pulse, and Z is the mJ/mb of the long pulse. In some embodiments, the minimal coagulation setting is 22-0-0.

### Application of Cells to Skin Explants

Following laser surgical treatment (within about 5-10 minutes), the skin was topically treated with CFDA-SE labelled HSF, or injected with HSF for positive controls, then the skin explants were surrounded with skin feeding medium (TB-1 clonal express lymphocyte differentiation media (Promocell, Heidelberg, Germany; C-78610, which is a serum-free, completely defined medium supplemented with albumin, transferrin, lecithin, ethanolamine, fatty acids, selenium, pyruvate, KNO₃, additional amino acids, vitamins and HEPES buffer) supplemented with 10% FBS, 20 mM L-glutamine, 20 mM Glutamax™, 2.5 μg/mL amphotericin-B, and 50 μg/mL gentamicin sulphate (Mediatech, Inc., Manassas, Va.)). Media was permitted to touch the skin, but not cover it (the surface of the skin remained dry).

CFDA-SE labelled fibroblasts were topically applied at a volume of 100 μL per treatment area using a 30 G needle (BD, Franklin Lakes, N.J.). The fibroblasts were not permitted to reach the edges of the skin or mix with the skin feeding media.

A 1.0 cc disposable plastic syringe fitted with a 30 gauge ADG (Adjustable Depth Gauge) needle was used to inject fibroblasts, so that the depth was essentially the same for each injection (to reach mid-dermis) and similar injection speed/pressure as well as similar volume of 0.1 cc was used for each injection.

Skin was placed in an incubator at 37° C. and 5% CO₂ for 45-60 min to allow fibroblast adhesion to the skin. This time was chosen based on prior observations which showed that fibroblasts adhered to a cell culture vessel within 45 minutes. The skin was left uncovered during this incubation.

Following incubation and adhesion, 4 mm punch biopsies were taken and placed in 10% buffered formalin. Tissue biopsies were sent to Dominion Pathology (Norfolk, Va.) for paraffin embedding, sectioning, mounting, and staining. Histological slides were stained with either Massons' Trichrome or left unstained (for fluorescence imaging).

### Imaging & Data Analysis

Imaging was performed on a Leica DM-IRB microscope equipped for fluorescence. Fluorescence excitation and emission was achieved using a mercury bulb light source (HBO100 Arclamp, LED Ltd.) passing through a Chroma Technology FITC filter set. A DAPI filter set was used for the UV images; the Masson images used visible light and the CFSE used the previously mentioned FITC set (standard green fluorescence). Images were acquired at 10× magnification (Leica 10× N-plan, 0.25 N.A.; Leica Microsystems Inc., Buffalo, N.Y.) using an Optronics Magnafire CCD camera (Optronics, Goleta, Calif.) with 20.614 s exposure time (fluorescence) or a 4.419 ms exposure time (optical). Image acquisition was conducted via both Magnafire v.2.1A combined with ImagePro Plus (v.4.5.1.23, Media Cybernetics, Inc., Bethesda, Md.). Serial images were acquired using approximately 20% overlap between images to allow compositing. Images were composited using Adobe Photoshop (version 6.0, Adobe Systems Inc., San Jose, Calif.).

### Results and Analysis

In this example, human skin fibroblasts (HSF) were grown to confluence and topically applied to human skin explants that had been treated with various dermatologic lasers in order to produce voids in the skin tissue.

For successful delivery and survival of live cells, it is important that they are able to penetrate in sufficient numbers into the voids, that they then are not killed by any substance (e.g., an inflammatory or other response) within the voids or engendered by immediately adjacent tissue. In general, the cells are provided with voids of sufficient diameter to allow entry, voids that are ‘empty’ to allow penetration, and that are of sufficient depth to allow cells to reach the intended target ‘home’ within the target tissue (e.g., the area either where they natively exist or where they are therapeutically targeted). The cells must then be provided with an environment that enables them to survive and preferably thrive, which means not having a hostile cellular environment either immediately or longer term in that region.

Surprisingly, it was found that delivery of live fibroblast cells into laser-formed voids in skin explants required that the laser setting was minimally coagulating to the tissue.

Tables 3 and 4 show the number of holes and cells observed in a single imaged field of the biopsied tissue for each treatment, under a 10× objective lens. The same volume of cells was applied either topically or injected. The numbers are counts for injected and cells counted inside laser holes for topically applied samples. It is noted that the spacing and density of holes does vary by laser, as well as across each tissue sample treated with the lasers. Thus, it is recognized that the samples are homogenous. However, it is clear from the data in Table 3 that only one laser treatment (using the Palomar StarLux® 500 Erbium:YAG 2940 nm laser in a minimally coagulating setting) yielded voids in the target tissue into which fibroblasts readily were delivered using passive surface application.

TABLE 3

| Topically applied HSF | Holes | Cells |
|---|---|---|
| Fraxel Thulium | 2 | 0 |
| Fraxel Erbium* | 0 | 0 |
| Palomar Coagulated | 6 | 3 |
| Palomar Min-Coagulated | 2 | 40 |
| Palovia | 4 | 0 |
| Fraxel CO₂ | 2 | 0 |

*No laser holes were visible in the one examined field; not every slide or field was examined.

TABLE 4

| Injected HSF | Holes | Cells |
|---|---|---|
| Fraxel Thulium | 1 | 335 |
| Fraxel Erbium | 6 | 304 |
| Palomar Coagulated | 1 | 262 |
| Palomar Min-Coagulated | 1 | 244 |
| Palovia | 3 | 39 |
| Fraxel CO₂ | 1 | 3 |

This Example illustrates the lack of or minimal penetration into skin tissue of human skin fibroblasts (HSF) with all tested devices but for the Palomar 2940 in minimal coagulation mode.

The work described herein examined the feasibility of transepidermal delivery of topically applied autologous living human skin fibroblasts with fractional laser treatments. Culture grown CFSE (green fluorescent dye) treated human skin fibroblasts were applied post laser treatment to ex vivo human skin. Biopsies were examined under a fluorescent microscope determined the quantity of CFSE stained cells in the dermis.

Histological examination documented distinctive patterns of microchannel voids of varying depth and diameter for each laser. Intradermally injected fibroblasts were readily observed by fluorescence microscopy following treatment with all lasers, although fewer viable cells were observed in skin treated with the 1410 nm diode and CO₂ lasers. In contrast, topically-applied cells penetrated and adhered only in microchannels created by the Erbium 2940 nm laser with minimal coagulation (varying optics still show similar results). Topically-applied cells persisted and remained viable for up to 48 hours as demonstrated by serial biopsy. Thus, transepidermal delivery of viable human skin fibroblasts was demonstrated using minimally coagulative fractional 2940 nm laser parameters but not with the other tested lasers and laser parameters.

### Example 2

### Transepidermal Water Loss (TEWL) Test following Skin Treatment with Palomar StarLux 500 2940 nm Laser

TEWL correlates with disruption or damage to the skin's epidermal barrier layer; that is, it indicates removal of some impediment to increased permeability of the skin. This example demonstrates, using TEWL analysis, that treatment of live in vivo human skin with the 2940 nm Palomar StarLux Series 500 laser at select minimally coagulating settings results in measurable water loss, minimal bleeding, and rapid recovery.

### Materials & Methods

The Palomar StarLux 500 (2940 nm, Green Optic) laser was tested on a human subject's inner forearm at two settings: 1) 24 mJ, 850 micron depth, 2% density and 180 MTZ/cm² (single pulse), and 2) 12 mJ, 850 micron depth, 2% density and 180 MTZ/cm² (double pulse). The area to be treated was imaged with high resolution digital camera and close up (macro) lens. A single baseline TEWL measurement was taken using the Cortex Technology Dermalab with the Trans Epidermal Water Loss probe. The measurement settings took a continuous reading for 30 seconds on each treated area before compiling a final value of water loss.

Following laser treatment, the areas were treated according to standard of care for laser resurfacing, which included antibiotic ointment and occlusive dressings for three days. The post treatment measurements and images were taken using the equipment previously described.

TABLE 5

TEWL

| Palomar Lux500 | Pre | Immediately | 72 hrs Post |
|---|---|---|---|
| 2940 nm Settings | treatment | Post treatment | treatment |
| (24-0-0) | 0.9 | 65.6 | 8.3 |
| single pulse | | | |
| (12-0-0, 5.0/Hz) | N/A | 68 | 0.0 |
| double pulse | | | |

The TEWL results demonstrate a significantly increased loss of moisture of approximately the same magnitude immediately following both treatment settings. The 72 hour data demonstrates that the 12 mJ setting has TEWL measurements similar to baseline, while the 24 mJ setting still displays a slight increase in moisture loss. The setting used resulted in minimal to no visible bleeding from the subject.

### Example 3

### Delivery of Live Human Skin Fibroblasts to Skin Tissue

This example corroborates data presented in Example 1, and further supports the conclusion that treatment of live human skin explants with the 2940 nm Palomar Star Lux 500 2940 nm laser at a minimally coagulating setting

### Methods and Materials

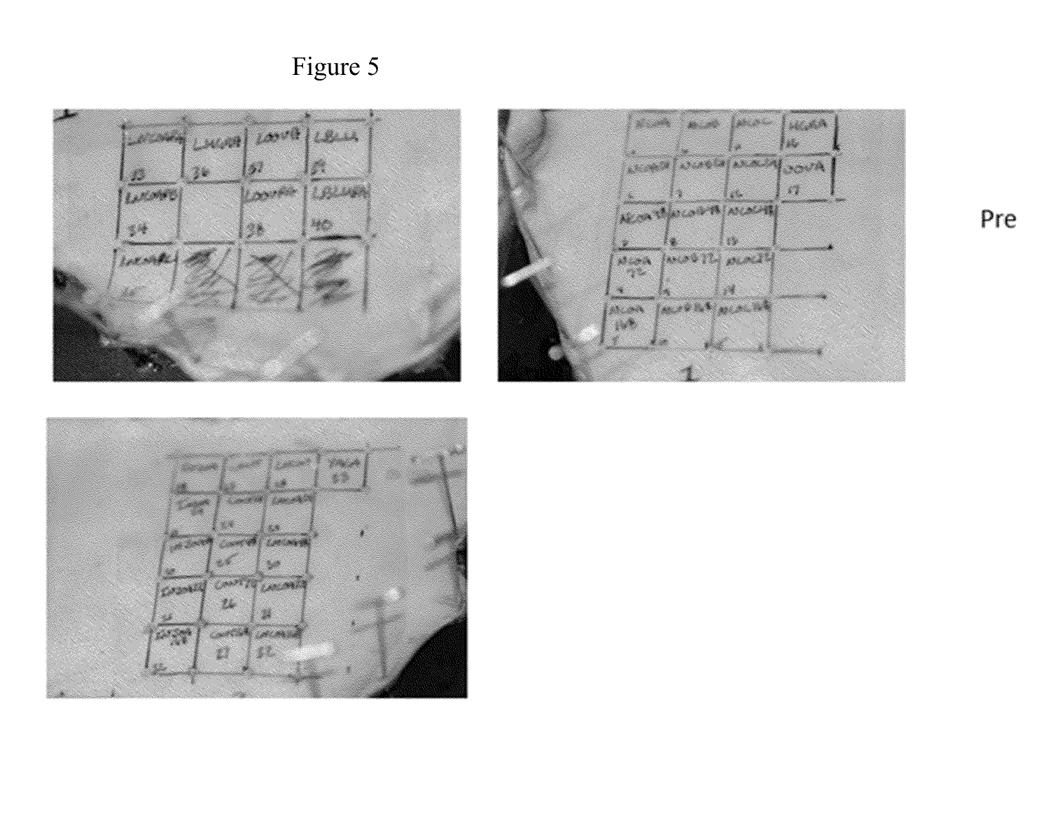

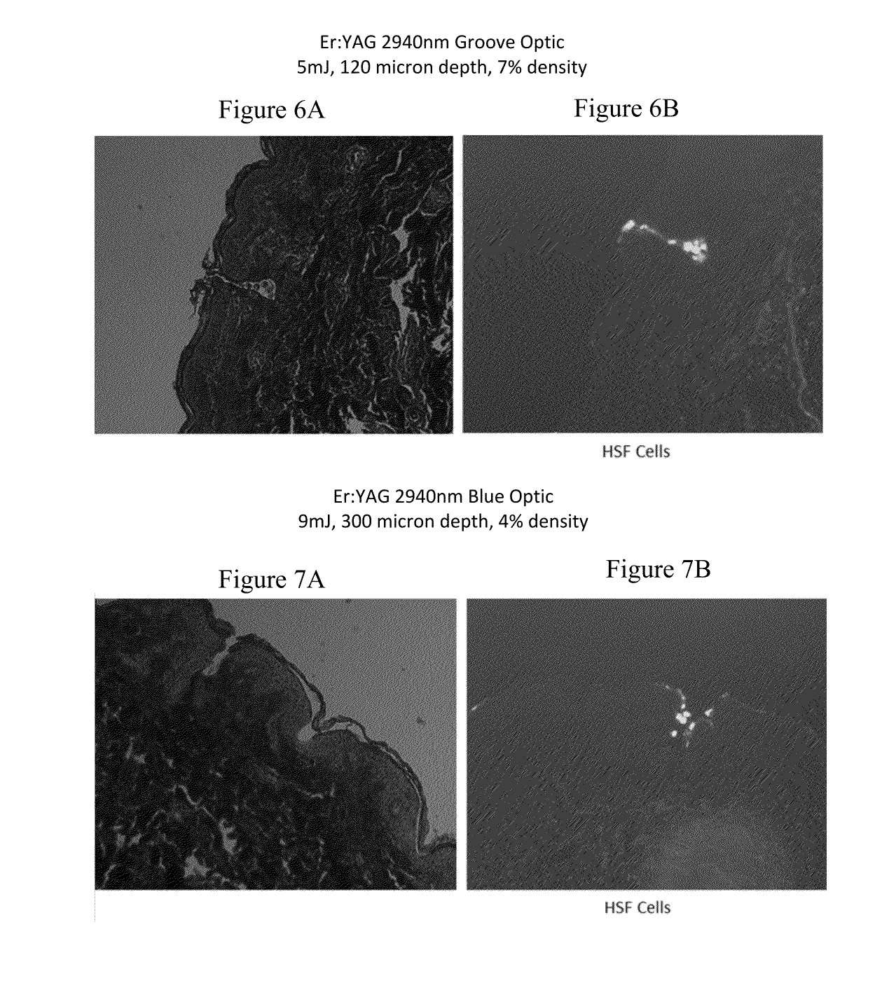

The skin explants were treated in the same fashion as for Example 1. Briefly, they were excised from a human living body and placed on ice in sterile saline solution for transport to the lab. Once at the lab, the explants were removed from saline and defatted using a Personna Plus sterile surgeon's blade 10S. The skin was then laid out in a dissecting tray and pinned under some tension using T Pins. The skin was then gridded as previously described (FIG. 5) and treated with each laser in sequence. The topical cells and/or injections (100 μL, of labelled cell suspension) were then applied after all laser treatments, essentially as described in Example 1.

TABLE 6
LASER + HSF PARAMETERS

| Elapsed Time | Palomar Er:YAG | | | Palomar Er:YAG |
|---|---|---|---|---|
| between | minimal Coag (2940 nm) | | | minimal Coag (2940 nm) |
| Application | Green Optic, 24 mJ, 850 micron | | | Green Optic 24 mJ, 850 micron |
| of Cells and | depth, density 2%, 180 MTZ/cm | | | depth, density 2%, 180 MTZ/cm2 |
| Biopsy | 224-0-0* + HSF | | | 12-0-0* + HSF |
| 45 min-1 hr | NCOA | NCOB | NCOC | HGRA |
| 24 hr | NCOA 24 | NCOB 24 | NCOC 24 | Palomar Er:YAG minimal |
| | | | | Coag (2940 nm) Groove Optic |
| | | | | Handpiece 5 mJ, 120 micron depth, |
| | | | | density 7%, 5-0-0* + HSF |
| 48 hr | NCOA 48 | NCOB 48 | NCOC 48 | OOVA |
| 72 hr | NCOA 72 | NCOB 72 | NCOC 72 | Palomar Er:YAG minimal |
| | | | | Coag (2940 nm) BLUE Optic, |
| | | | | 9 mJ, 300 micron depth, 4% |
| | | | | density (1 hr only) 9-0-0* + HSF |
| 168 hr | NCOA 168 | NCOB 168 | NCOC 168 | HBLU |

*See below, with Table 7.

TABLE 7
LASER AND CONTROL PARAMETERS

| | | LASER ONLY (no HSF) | |
|---|---|---|---|
| | | Palomar Er:YAG | |
| | | minimal Coag (2940 nm) Green | 1064 nm YAG |
| | | Optic, 24 mJ, 850 micron depth, | laser, 13.5 J/cm², |
| | Untreated | density 2%, 180 MTZ/cm2 | 2.0 mm spot, |
| Injected HSF Only | Controls | 224-0-0* | single pulse |
| INJOA | CONT | LNCOA | YAGA |
| INJOA 24 hrs | CONT 24 hrs | LNCOA 24 hrs | |
| INJOA 48 hrs | CONT 48 hrs | LNCOA 48 hrs | |
| INJOA 72 hrs | CONT 72 hrs | LNCOA 72 hrs | |
| INJOA 168 hrs | CONT 168 hrs | LNCOA 168 hrs | |

*Instrument settings for 2940 nm laser: x-y-z, where X is the mJ/mb @250 microsecond (which is the duration of the short pulse), Y is the pulse duration of the long pulse in milliseconds, and Z is the mJ/mb of the long pulse.

### Results

As with Example 1, this example provided data showing that topically-applied cells persisted and remained viable when applied to ex vivo human skin pre-treated with minimally coagulative fractional 2940 nm laser parameters. Notably, the duplicate samples described in this example provided evidence that the technique is repeatable.

### Example 4

### Analysis of Protein and/or Expression in Target Tissue after Void Creation

This example provides a representative method for analyzing protein expression and changes of protein expression in human tissue after void generation with the 2940 nm Palomar StarLux 500 laser. Similarly, gene expression changes can be analyzed, for instance using PCR or other recognized nucleic acid analysis techniques.

### Methods and Materials

Samples are prepared essentially as described above (though optionally the skin or other tissue biopsies may be taken from live subjects as well as from explants). By way of example, protein array analysis tissue harvested is from a unique 4 mm punch away from the edges of any histology punches. By way of example, the skin tissue is subject to the following laser treatments prior to tissue sampling:

TABLE 8
Palomar Er:YAG NO Coag (2940 nm), sampled 1 hour after laser treatment

| Green Optic, 24 mJ, | Green Optic, 12 mJ, | | |
|---|---|---|---|
| 850 micron depth, | 850 micron depth, | Groove Optic Handpiece | BLUE Optic, 9 mJ, |
| density 2%, 180 | density 2%, 180 | 5 mJ, 120 micron depth, | 300 micron depth, 4% |
| MTZ/cm2 24-0-0* | MTZ/cm2 12-0-0* | density 7%, 5-0-0* | density 9-0-0* |
| LNCOAP rep A | LHGRA | LOOVA | LBLU |
| LNCOAP rep B | | LOOVA PA | LBLU PA |
| LNCOAP rep C | | | |

*As for Tables 6 & 7.

### Protein Extraction for Protein Array Analysis

Protein samples are prepared in accordance with RayBiotech Protein Cytokine Array instructions, with minor modifications as follows. Previously biopsied and flash frozen skin samples are thawed, blotted dry with a Kimwipe® tissue, and 100 mg of tissue is excised beginning with the tissue closest to the initial biopsy (used for histology and centered on the laser treated area). This first sample is taken on all laser treated samples and controls. The tissue samples are then placed in Ray Biotech Cell Lysis Buffer that is supplemented with Protease Inhibitor Cocktail III (EDTA-free) at a 5 μL stock solution (per 100 mg tissue, based on manufacturer's guidelines) diluted in Li-Cor Blocking Buffer (supplemented w/0.01% Tween 20) to yield 1 mL of Lysis Buffer. The tissue is homogenized in the buffer using a manual, glass 7 mL Dounce homogenizer (first three samples) or a handheld Kontes pestle mortar with disposable tips.

Alternatively, samples are homogenized by immersion in liquid nitrogen and grinding in a pre-chilled ceramic mortar and pestle. The resulting crystallized skin and lysis buffer “snow” is collected in a microfuge tube and thawed in a 37° C. water bath. Skin is traditionally difficult to homogenize, and thawed, room-temperature techniques require violent dissociation means that disrupt protein structure. Such techniques are not suitable for antibody-based detection methods, which rely on intact protein (antigen) conformation.

The solutions are centrifuged for 10 minutes@10,000×g and the supernatant assayed for total protein content using a Pierce BCA Assay.

Aliquots of the resultant supernatant are prepared at 250 μg/mL using a slightly modified version of the LiCor Odyssey system protocol (available on-line at biosupport.licor.com/support posted as a revision of July 2008).

By way of examples, the arrays analyzed are RayBiotech AAH-INF-3-8, and they detect: Eotaxin, Eotaxin-2, GCSF, GM-CSF, ICAM-1, IFN-gamma, I-309, IL-1 alpha, IL-1 beta, IL-2, IL-3, IL-4, IL-6, IL-6sR, IL-7, IL-8, IL-10, IL-11, IL-12p40, IL-12p70, IL-13, IL-15, IL-16, IL-17, IP-10, MCP-1, MCP-2, M-CSF, MIG, MIP-1 alpha, MIP-1 beta, MIP-1 delta, RANTES, TGF-beta1, TNF-alpha, TNF-beta, sTNF RI, sTNF-RII, PDGF-BB, TIMP-2.

The membranes are pre-incubated for 30 minutes at room temperature in Li-Cor Odyssey blocking buffer supplemented with 0.01% Tween 20. The membranes are then drained and 1 mL of sample is applied to each membrane and placed on a shaker platform on slow speed to incubate at room temperature for 2 hours. At that time the membranes are washed 3× using 2 mL of washing buffer I for each wash (Ray Biotech provided in kit). The membranes then get a second series of washes using washing buffer II (Ray Biotech provided in kit) again using 2 mL per wash for two washes.

The detection antibodies are reconstituted in 100 μL of blocking buffer and then further diluted to 2 mL of volume using additional blocking buffer. This is added to the membranes with 1 mL of detection antibody solution for each. The membranes are incubated at 4° C. overnight. After incubation the membranes are again washed as previously described (3× with washing buffer I and 2× with washing buffer II). The Streptavidin-IR 800CW detection solution is diluted to a concentration of 1 μg/mL and 2 mL is added to each membrane. The membranes are then incubated for 2 hours at room temperature wrapped in foil to protect from light. After 2 hours the membranes are washed for 5 minutes each with washing buffer I (four washes) and washing buffer II (two washes). The membranes are then stored until scanning in washing buffer II at 4° C.

Genes or proteins that show statistically different expression levels between untreated and laser treated skin/tissue, or between different laser treatments, can be selected for further study. Alternatively, laser treatments can be tuned based on gene/protein expression profiles, for instance in order to tailor the laser treatment in order to minimize potentially damaging (to the implanted cells and/or the surrounding tissue) expression and/or increasing expression of beneficial genes or proteins.

### Example 5

### Analysis of Common Laser Pre-Treatments on in Vitro Cultivated Fibroblasts

This example describes in vitro (in silico) testing of compounds added to human fibroblast cell cultures to determine any detrimental effects to the cells.

Human fibroblast cells in culture (prepared essentially as described for Example 1) were incubated with the listed compounds for 45 minutes (estimated as about the time exogenously-applied fibroblast cells would be in contact with any residue on skin in herein-described treatments), then visually inspected. The cells were then rinsed and placed in fresh media, and observed again at 24 hours and one week post exposure. The results (based on visual inspection) are provided in Table 9.

TABLE 9

| | Viability/nspection | Viability/Inspection | Viability/Inspector |
|---|---|---|---|
| Parameter tested | 45 min | 24 hrs | 1 week |
| Epinephrine 1:100,000 | Slight change in morphology, | Normal | Confluent/overgrown |
| | narrowing | | |
| Epinephrine 1:10,000,000 | Normal | Normal | Confluent/overgrown |
| Lidocaine 0.5% | Normal | Normal | Confluent/overgrown |
| Lidocaine .01% | Normal | Normal | Confluent/overgrown |
| Epi 1:100,000 + 1% Lido | Normal | Normal | Confluent/overgrown |
| Epi 1:10,000,00 + 0.1% Lido | Normal | Normal | Confluent/overgrown |
| Lidocaine Cream 1% | Holes in monolayer | Still losing cells, larger holes | Confluent/overgrown |
| Lidocaine Lotion 1% | Small holes in monolayer | Holes present with slight | Confluent |
| | | regrowth/filling in | |
| .01% Lido + Sodium | Very large holes and some | Large amount of sheeting and | Primarilly dead, no regrowth |
| B carbonate | sheeting of monolayer | cellular debris | |
| (Physiologic) | | | |
| Epi 1:100,000 + Sodum | Lot of disruption to monolayer | Large patches of sheeting and | Primarilly dead, no regrowth |
| B carbonate | and cellular debris | cell death | |
| (Physiologic) | | | |
| SRF + petrolatum 1% | Normal | Normal | Confluent/overgrown |
| SRF [symbol] lipoderm 1% | Minor holes in monolayer | Normal | Confluent/overgrown |
| Effacil Cleanser 5% | Massive holes in monolayer | Cells gone | N/A |
| Effacil Cleanser 1% | Unhealthy appearance, small | Major holes and sheeting | Cells gone |
| | holes in monolayer | | |
| 1:2 Vinegar Water | Normal | Shriveled cell appearance and | Normal |
| | | debris | |
| 1:40 Vinegar Water | Normal | Normal | Confluent/overgrown |
| Control x2 | Normal | Normal | Confluent/overgrown |

[symbol]

indicates data missing or illegible when filed

“SRF” as it used in the above table and herein refers to Skin Resuscitation Factor™ (SRF™) from NorthCell Pharmaceutical (U.S. Pat. No. 7,122,578). It is a petrolatum-based formula that includes sodium pyruvate, alpha keto isovalerate, keto butyrate, keto glutarate, keto caproate, keto adipate, and oxaloacetate and is designed to reduce inflammation, aid wound healing and eliminate peroxynitrite production. The remaining compositions and mixtures were tested as they are expected to be common treatments that might be used for numbing or cleansing of skin prior to laser treatment and cell delivery, or coagulation compounds, that are available in most physician offices. As such, these compounds are believed likely to be present on skin prior to or during treatment with the cell delivery methods described herein, where they interfere with the efficacy of this procedure. In addition, the delivery vehicle for SRF and lidocaine were tested to determine which vehicle (cream, lotion, petrolatum, lipoderm) was least destructive to the cells.

Based on these results, Effacil® cleanser and sodium bicarbonate are clearly undesirable in preparation of tissue (for instance skin) that is to be treated using the cell delivery methods described herein. These compounds showed large amounts of cell lysis (large holes in the confluent monolayer) and in most cases the cells were not able to recover in 24 hours or even a week. These compounds were judged to be too harsh and most likely fatal to the topically applied fibroblasts, and if used on the skin (and any traces remained on the skin to contact the topically applied cells) a large percentage of the cells would die before achieving penetration and attachment. It is believed that any product that demonstrated lasting (past 24 hours) cellular damage should be avoided.

### Example 6

### Methods for Fractional Laser Delivery of Topically Applied Autologous Human Skin Fibroblasts

A fractional 2940 nm Er:YAG laser was used with minimal coagulation parameters followed by topical application of cells to treat the full face or full face, neck and chest in two patients. All patients were followed with digital imaging.

Patents were provided the following pretreatment instructions for the time (1-2 weeks) immediately prior to the procedure:
1. Discontinue use of Fish oil, Omega 3, baby aspirin (unless aspirin is required by physician), Retin A or other retinoids.
2. No peels/chemical facials for two week prior to the procedure.
3. Use SkinCeuticals CE Ferulic® combination antioxidant treatment (or equivalent non-cytotoxic antioxidant formulation, for example, an antioxidant formulation comprising ascorbic acid, tocopherol, and ferulic acid) product once daily, applied to the areas to be treated, for two weeks prior to the procedure.
4. Apply SkinCeuticals Physical Fusion UV Defense SPF50 sunscreen daily to the areas to be treated, for two weeks prior to the procedure.
5. Test SRF™ ointment on skin in a small area for 2-3 days at least one week prior to the procedure (for instance, by applying a thin film to the forearm or behind one ear for 2-3 nights).
6. Use SkinCeuticals Gentle Cleanser as the only face/skin tissue for the area to be treated for at least a week prior to treatment.
7. If the face is being treated, take an antiviral agent (usually acyclovir) orally for 1-2 days prior to the procedure and for a few days afterward (exact instructions are provided on specific prescription).
8. Avoid excessive sun exposure, sunburn, or tanning beds for 1 week before your procedure

The following day-of-procedure protocol was employed for facial treatment:
1. Patient arrives at office with no makeup or skin care lotion on face and/or areas to be treated.
2. Patient fills out and signs consent form, and optionally photo release.
3. Patient is given one ibuprofen only.
4. “Pre-treatment” photos are taken, if not done at Pre-op visit.
5. Patient's face is cleansed using Clarisonic® sonic skin cleansing brush on low setting using the SkinCeuticals® Gentle cleanser using the sensitive brush.
6. Optionally, perform one treatment of GentleWaves® skin treatment yellow to all areas to be treated.
7. Apply 30% lidocaine ointment for numbing, 30 minutes.
8. Cleanse face using Clarisonic® skin cleansing brush on gentle setting using the SkinCeuticals® Gentle Cleanser or equivalent (such as a non-cytotoxic cleanser). Ensure all lidocaine ointment is off before proceeding.
9. Mark the face with the patient sitting up, using a washable style marker.
10. Eye shields are inserted (if treating under eyes); otherwise microderm “stickies” are used if full face treatment, or use gauze pads.
11. Treat the skin using the 2940 nm Palomar laser and Blue handpiece on setting 5/0/0 with a repetition rate of about five around eyes and typically about three elsewhere, then to Green handpiece at 22/0/0. Holding the laser sideways for better vision and stability, treat the areas in a tile-laying manner with 0-10% overlap and one pass. Stretch skin where needed to obtain complete coverage.
12. For treatment, the face is divided into thirds, for instance, in the following order: 1. Left side of face to midline from jaw to brow; 2. Right side of face to midline from jaw to brow; and 3. Full forehead. Prepared autologous fibroblast cells are applied to skin after each section is finished; do not wait to apply until the entire face is treated. Clean the laser optics after each section.
13. While treating, keep the vacuum close to the skin and wipe the optical window frequently.
14. It is acceptable to use ice on the skin surface before applying cells; however, do not apply ice after cells have been applied.
15. To prevent bleeding: Blot (but do not wipe) bleeding area with dry gauze. If needed, 1:100,000 epinephrine may be applied to the skin, for instance using a cotton swab to only the spot(s) that is bleeding.
16. Apply autologous cells topically to the treated area via 1.0 cc syringe with needle removed, and gently massage in with sterile gloved hands to each zone/segment treated (in case of face, one third of the skin in the order in which it was treated); then apply second application of cells 1-2 minutes after first application of cells. Estimated volume for full face is approximately 1 cc per coating. Optionally: apply one coat of patient's “conditioned media extract” and invert the cells gently before using them for treatment. Optionally: Treat cell-covered areas using positive pressure, for instance using a device that applies gentle positive gas pressure such as the “positive pressure syringe” device (FIG. 8A), to encourage cells into perforations in the skin.
17. Apply Skin Resuscitation Factor™ (SRF™) anti-inflammatory ointment 1-2 minutes after the second application of cells. If doing treatment in stages of anatomic units, be careful to not get SRF™ ointment on the yet-to-be-treated areas; leave a small margin not covered with SRF™ and overlap later. If SRF™ ointment not used, apply plain petrolatum in its place
18. Review aftercare instructions (see below) with patient; set up follow up appointments and confirm patient has aftercare supplies. Typically, follow up appointments occur at 3 days, one month and six months post treatment.
29. Take immediate post treatment photos (optional).

The above procedure is provided in the context of face or neck treatment. The striae or stretchmark protocol is essentially the same, with minor technique changes based on the treatment location. Specifically, the cells are rubbed in after the entire area has been treated and then covered with SRF or petrolatum.

The following post treatment (aftercare) instructions were explained to each patient:

Wash your face (or other treated area) only with the sterile saline solution provided until the 3^{rd} evening after your treatment. After this time you may use daily SkinCeuticals Gentle Cleanser (or equivalent, such as a non-cytotoxic cleanser). Use SRF™ ointment twice daily for 3-7 days after your laser treatment; you may stop as soon as healing has occurred, but should apply the ointment a minimum of three days.

Use SkinCeuticals Physical Fusion UV Defense SPF50 and begin to apply on the 3rd or 4th day after your procedure. Note: Until SPF can be applied, avoid direct exposure to sunlight (or any other source of UV light). Apply the SkinCeuticals Physical Fusion UV Defense SPF50 directly over the top of the SRF™ ointment. If your face was treated with the 2940 nm fractional laser, continue your antiviral Rx for as many days as directed on your prescription. You may begin to apply makeup 5-7 days post treatment, but may be sooner depending on doctor evaluation and advice.

Expect mild to moderate swelling, redness, puffiness or bruising following treatment. Contact doctor for any questions or if you experience persistent or unexpected side effects.

Do not apply ice to the treated skin after treatment. Do not use tanning beds. Avoid excessive sun exposure and sunburns for the first 4-6 weeks after the procedure. Avoid all sunlight/UV exposure for 48 hours after the treatment/procedure. Do not rub, scrub or manipulate the treated areas for 24 hours. Do not shave the treated areas for 24 hours post treatment or until your skin has healed. Do not use Retin A, Atralin, Differin, Tazorac, Obagi, tretinoin, etc., anti-aging products, Rx products, or other physician dispensed products that have not been approved by the treating physician for four weeks after treatment. The following products are approved for use in the first week post treatment: SkinCeuticals Gentle Cleanser (or equivalent, such as a non-cytotoxic cleanser), SkinCeuticals CE Ferulic™ (or equivalent non-cytotoxic antioxidant formulation, for example, an antioxidant formulation comprising ascorbic acid, tocopherol, and ferulic acid), SkinCeuticals Physical Fusion UV Defense SPF50, Antiviral Rx, sterile saline, and SRF™ formula in Petrolatum. Do not have a facial, microdermabrasion, chemical peel or laser or light treatment (excluding GentleWaves) for four weeks after treatment unless approved by treating physician.

The aftercare instructions for striae are modified as follows:
The following post treatment (aftercare) instructions were explained to each patient:

Wash your treated area only with the sterile saline solution provided until the 3^{rd} evening after your treatment. After this time you may use daily SkinCeuticals Gentle Cleanser (or equivalent, such as a non-cytotoxic cleanser). Use SRF™ ointment twice daily for 3-7 days after your laser treatment; you may stop as soon as healing has occurred, but should apply the ointment a minimum of three days.

Use SkinCeuticals Physical Fusion UV Defense SPF50 and begin to apply on the 3rd or 4th day after your procedure. Note: Until SPF can be applied, avoid direct exposure to sunlight (or any other source of UV light). Apply the SkinCeuticals Physical Fusion UV Defense SPF50 directly over the top of the SRF™ ointment.

Expect mild to moderate swelling, redness, puffiness or bruising following treatment. Contact doctor for any questions or if you experience persistent or unexpected side effects.

Do not apply ice to the treated skin after treatment. Do not use tanning beds. Avoid excessive sun exposure and sunburns for the first 4-6 weeks after the procedure. Avoid all sunlight/UV exposure for 48 hours after the treatment/procedure. Do not rub, scrub or manipulate the treated areas for 24 hours. Do not shave the treated areas for 24 hours post treatment or until your skin has healed. Do not use Retin A, Atralin, Differin, Tazorac, Obagi, tretinoin, etc., anti-aging products, Rx products, or other physician dispensed products that have not been approved by the treating physician for four weeks after treatment. The following products are approved for use in the first week post treatment: SkinCeuticals Gentle Cleanser (or equivalent, such as a non-cytotoxic cleanser), SkinCeuticals™ (or equivalent non-cytotoxic antioxidant formulation, for example, an antioxidant formulation comprising ascorbic acid, tocopherol, and ferulic acid), SkinCeuticals Physical Fusion UV Defense SPF50, sterile saline, and SRF™ formula in Petrolatum. Do not have microdermabrasion, chemical peel or laser or light treatment (excluding GentleWaves) for four weeks after treatment unless approved by treating physician.

### Example 7

### Use of Fractional Laser Delivery of Topically Applied Autologous Human Skin Fibroblasts to Human Patients—Face, Neck, and Chest

For this study, employing the procedures described in Example 6, live autologous fibroblast cells were used to treat patients for the reduction of wrinkles in the skin. Patients were treated with fractional 2940 nm laser and topically applied autologous skin fibroblasts, then followed clinically.

### Face and Neck Cases

Subjects treatment was completed as described above using four total vials of cells to cover the full face, neck and chest following laser treatment. The subjects neck and chest was also completed using the same general protocol as previously described for the face, with the exception of dividing the neck and chest into halves and not thirds. The aftercare was identical with two topical applications of cells in suspension several minutes apart and application of SRF™ ointment as the final step. A second subject had the only the face treated as described above and used a total of two vials of cells.

### Results:

The patients tolerated the procedure well with no adverse events. Significant wrinkle reduction was observed at one month post treatment for the face, neck and test and at three months post treatment for the face in another patient. The initial treatments of face, neck and chest appeared to have more rapid wound healing and reduced erythema compared to the inventor's experience with the laser alone.

Additional, longer term follow up is expected to show improvements to fine lines and wrinkles, possible skin tightening effects as well as filler type effects that persist for longer than “normal” fillers. The filler effects may be more subtle, but are believed to be significantly longer lived (possibly permanent). Based on observations so far, the other effect is increased “glow” or vibrance to the skin.

Fractional laser assisted topical delivery of autologous skin fibroblasts is a viable clinical technique with potentially unique benefits and also technical challenges for treating photoaging.

### Example 8

### Use of Fractional Laser Delivery of Topically Applied Autologous Human Skin Fibroblasts to Human Patients—Striae (Stretchmarks)

For this study, employing the procedures described in Example 6, live autologous fibroblast cells will be used to treat patients for the reduction of striae in the skin. Patients will be treated with fractional 2940 nm laser and topically applied autologous skin fibroblasts, then followed clinically.

### Stretch Mark Case

In this case, one striae alba patient is to receive both the 2940 nm laser alone and in combination with topically applied cells and also for comparison, injected cells only, 1540 nm fractional laser alone and in combination with topically applied cells and a no treatment control. One abdominoplasty scar patient is to receive the same striae protocol.

It is expected there will be no adverse events and a significant reduction in the appearance of striae will be seen with both fractional laser treatments. It is also anticipated that the addition of topically applied cells will improve both the appearance of the striae and possible the underlying dermal matrix as well.

### Example 9

### Use of Fractional Laser Delivery of Topically Applied Autologous Human Skin Fibroblasts to Human Patients—Upper Lip

For this study, employing the procedures described in Example 6, live autologous fibroblast cells are used to treat patients for the reduction of wrinkles in the skin on the upper lip. Patients are treated with fractional 2940 nm laser and half the lips are treated with topically applied autologous skin fibroblasts (approximately 15-20 million cells per vial, for example 18 million cells per vial), then followed clinically.

Two upper lip patients are being treated in this study, with the full lip receiving laser and only one side of each subject's lip will receive topically applied cells. At least the following metrics will be examined: Wrinkle reduction (and duration thereof), redness and its duration, days to scabbing or other healing metrics. There will be the same set of aftercare and pre care instructions.

This split upper lip treatment study will provide insight into the role of the topical fibroblast application, and specifically how much of the observed beneficial effects depend on application of the autologous cell. It is expected that there will be a visible difference from one side of the lip to the other, solely based on the activity of the adherent cells.

While only certain embodiments have been described, it will be understood by those of ordinary skill in the art that various changes in form and details may be made therein without departing from the spirit and scope as defined by the claims. Those of ordinary skill in the art will recognize, or be able to ascertain using no more than routine experimentation, many equivalents to the specific embodiments described herein. Such equivalents are intended to be encompassed in the scope of the claims, and I therefore claim as my invention all that comes within the scope and spirit of these claims.

I claim:

1. A method of delivery of at least one live animal cell into tissue comprising:
ablating a portion of the tissue with electromagnetic radiation to form a plurality of voids extending to a depth below a surface of the tissue; and
delivering a live animal cell into one or more of the voids.

2. The method of claim 1, wherein delivering a live animal cell into the one or more voids comprises applying a composition comprising the cell onto the surface of the tissue.

3. The method of claim 2, wherein delivering the live animal cell comprises applying positive pressure to the surface of the tissue concurrently with or after applying the composition.

4. The method of claim 1, wherein conditions selected for ablating the portion of the tissue minimize the coagulation zone of tissue damage.

5. The method of claim 1, wherein at least a portion of the voids extend to a depth in a range of approximately 0.1 μm to 10 mm.

6. The method of claim 1, wherein at least a portion of the voids extend to a depth of approximately the dermal-epidermal border, to a depth inside the dermal layer, to a depth inside the epidermal layer, to a depth inside the subcutaneous layer, or to a depth deeper than the subcutaneous layer.

7. The method of claim 1, wherein the tissue is skin tissue and wherein the voids extend from the surface of the skin tissue to the epidermis.

8. The method of claim 1, wherein the tissue is skin tissue and wherein the voids extend from the surface of the skin tissue to the dermis.

9. The method of claim 1, wherein the tissue is skin tissue and wherein the voids extend from the surface of the skin tissue to below the dermis.

10. The method of claim 1, wherein each of the voids has a width in a range of approximately 0.1 μm to 1 mm.

11. The method of claim 1, wherein each of the voids has a width of approximately 50 μm to 250 μm.

12. The method of claim 1, wherein the tissue is ablated with electromagnetic radiation having one or more wavelengths of between approximately 1,850 to 100,000 nanometers and with pulse widths of between approximately 1 femtosecond (1×10⁻¹⁵ s) to 10 milliseconds (10×10⁻³ s) with fluence in the range of from approximately 1 J/cm² to 300 J/cm².

13. The method of claim 1, wherein the tissue is ablated with electromagnetic radiation having one or more wavelengths of between approximately 2,200 to 5,000 nanometers.

14. The method of claim 1, wherein the tissue is ablated with electromagnetic radiation having one or more wavelengths of between approximately 190 to 320 nanometers with fluence in the range of from 1 J/cm² to 300 J/cm².

15. The method of claim 2, wherein the voids comprise channels or grooves.

16. The method of claim 1, wherein the tissue is human tissue.

17. The method of claim 15, wherein the human tissue in a living subject.

18. The method of claim 16, wherein the live animal cell is autologous to the subject.

19. The method of claim 1, wherein the cell is cultured in vitro prior to being applied to the tissue surface.

20. The method of claim 1, wherein the cell is a fibroblast, an integument cell, an adipocyte, a preadipocyte, a stem cell, an epithelial cell, a retinal cell, an immune function cell, a melanocyte or other pigment cell, a hair follicle cell, a keratinocyte, or a Langerhans cell.

21. The method of claim 1, wherein the cell is a muscle cell, a bone cell, a pancreatic cell, a cell of a mucosal membrane, a chondrocyte, a cell of the nervous system, a hormone secreting cell, an endocrine cell, an intestinal cell, or a germ cell.
